SEQUENCE LISTING (10 sequences)

SEQ ID NO: 1 - DNA, 20 bp, Artificial Sequence
  synthetic sequence
gccgagtcaggaacagccag

SEQ ID NO: 2 - DNA, 23 bp, Artificial Sequence
  synthetic sequence
tttcttcttcttcttgcccagga

SEQ ID NO: 3 - DNA, 21 bp, Artificial Sequence
  synthetic sequence
atggatgactccacagaaagg

SEQ ID NO: 4 - DNA, 19 bp, Artificial Sequence
  synthetic sequence
tggtagaaagacaccaccg

SEQ ID NO: 5 - DNA, 20 bp, Artificial Sequence
  synthetic sequence
atgcccttgctttggctgag

SEQ ID NO: 6 - DNA, 23 bp, Artificial Sequence
  synthetic sequence
ttcaagtgcagcatgtttaagat

SEQ ID NO: 7 - DNA, 21 bp, Artificial Sequence
  synthetic sequence
atggatgagtctgcaaagacc

SEQ ID NO: 8 - DNA, 20 bp, Artificial Sequence
  synthetic sequence
ggacatcgctgtgaaactgc

SEQ ID NO: 9 - protein, 655 aa, Mus musculus
MMKFTVVAAALLLLGAVRAEEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKRALSSQHQARIEIESFFEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSGGPPPTGEEDTSEKDEL

SEQ ID NO: 10 - protein, 553 aa, Mus musculus
MLSVRVAAAVARALPRRAGLVSKNALGSSFVGARNLHASNTRLQKTGTAEMSSILEERILGADTSVDLEETGRVLSIGDGIARVHGLRNVQAEEMVEFSSGLKGMSLNLEPDNVGVVVFGNDKLIKEGDVVKRTGAIVDVPVGEELLGRVVDALGNAIDGKGPIGSKTRRRVGLKAPGIIPRISVREPMQTGIKAVDSLVPIGRGQRELIIGDRQTGKTSIAIDTIINQKRFNDGTDEKKKLYCIYVAIGQKRSTVAQLVKRLTDADAMKYTIVVSATASDAAPLQYLAPYSGCSMGEYFRDNGKHALIIYDDLSKQAVAYRQMSLLLRRPPGREAYPGDVFYLHSRLLERAAKMNDSFGGGSLTALPVIETQAGDVSAYIPTNVISITDGQIFLETELFYKGIRPAINVGLSVSRVGSAAQTRAMKQVAGTMKLELAQYREVAAFAQFGSDLDAATQQLLSRGVRLTELLKQGQYSPMAIEEQVAVIYAGVRGYLDKLEPSKITKFENAFLSHVISQHQSLLGNIRSDGKISEQSDAKLKEIVTNFLAGFEP

(19) United States
(12) Patent Application Publication
(10) Pub. No.: US 2019/0111040 A1
(43) Pub. Date: Apr. 18, 2019

(54) IN VIVO AMELIORATION OF ENDOGENOUS ANTI-TUMOR AUTOANTIBODIES TARGETING SURFACE TUMOR ANTIGENS VIA LOW-DOSE P4N

(71) Applicants: THE JOHNS HOPKINS UNIVERSITY, Baltimore, MD (US); NATIONAL CHIAO TUNG UNIVERSITY, Taiwan (CN)

(72) Inventors: Ru Chih C. Huang, Baltimore, MD (US); David Mold, Baltimore, MD (US); Tiffany Jackson, Baltimore, MD (US); Yu-Ling Lin, Taiwan (CN); Kuang-Wen Liao, Taiwan (CN)

(21) Appl. No.: 16/090,288

(22) PCT Filed: Mar. 30, 2017
(86) PCT No.: PCT/US2017/024905

§ 371 Date: Oct. 1, 2018

Related U.S. Application Data
(60) Provisional application No. 62/317,189, filed on Apr. 1, 2016.

(51) Int. Cl. A61K 31/4545; A61K 45/06; A61P 35/00; A61P 37/02

(52) U.S. Cl. CPC A61K 31/4545; A61P 37/02; A61P 35/00; A61K 45/06

(57) ABSTRACT

In accordance with the present invention, the immunoregulatory activity of low doses of P₄N was investigated. Unlike previously described antitumor drugs, low dose P₄N, in doses of about 1 to 10 mg/kg, or at concentrations of about 10 to 100 nM, was surprisingly found to contribute to humoral immunity by raising the titers and activities of autoantibodies against GRP78 and F1F0 ATP synthase on the surface of CT26 cells, and inducing B cell proliferation and differentiation of plasma cells. Methods for inducing endogenous antitumor autoantibodies (EAA) in a subject having a neoplasia comprising administering to the subject an effective amount of the nordihydroguaiaretic acid (NDGA) derivative P4N, or salts, solvates and stereoisomers thereof, as well as methods for inducing B cell proliferation, inducing BAFF stimulated B cell proliferation, and suppressing or inhibition growth of a neoplasia are also provided.

### REFERENCE TO RELATED APPLICATIONS

This application claims the benefit of U.S. Provisional Patent Application No. 62/317,189, filed on Apr. 1, 2016, which is hereby incorporated by reference for all purposes as if fully set forth herein.

### INCORPORATION-BY-REFERENCE OF MATERIAL SUBMITTED ELECTRONICALLY

The instant application contains a Sequence Listing which has been submitted in ASCII format via EFS-Web and is hereby incorporated by reference in its entirety. Said ASCII copy, created on Mar. 24, 2017, is named P14094-02_ST25.txt and is 12,252 bytes in size.

### BACKGROUND OF THE INVENTION

Colorectal cancer (CRC) is the second most prevalent cancer in the western world and is also rapidly increasing in Asia (1). It is well-known that multiple genetic events involved in the development of this disease lead to the generation of tumor-associated antigens (TAAs) against which. CRC patients develop autoantibodies (2). More than one hundred TAAs have been identified by these endogenous antitumor autoantibodies (EAAs), including 78 kDa glucose regulated protein (GRP78, also known as binding immunoglobulin protein (BiP)), different mutant p53, carcinoembryonic antigen (CEA) and mucin 1 (MUC1) (2). These autoantibody signatures have been proposed as biomarkers in the early detection of CRC (3-5). Typically, these EAAs have not exerted a significant effect on tumor elimination, which may be due to the immune tolerance induction by the tumor (6, 7). However, extraction of EAAs from the sera of cancer patients to activate the humoral immune response against some malignant tumors has been considered. A few EAAs selected from patients, such as SC-1 (anti-CD55), PAM-1 (anti-CFR1; cysteine-rich fibroblast growth factor) and PAT-SM6 (anti-GRP78), directly act against tumors and effectively kill them via antibody-mediated cellular cytotoxicity (8). In addition, a natural human IgM anti-GPR78 autoantibody (PAT-SM6) selected from patients' sera against the cell surface GRP78 protein provides therapeutic effects for cancer patients (9, 10). Although the therapeutic effects of EAAs are ill-defined, these studies still display their potential for clinical therapy by activating humoral immune responses against cancer.

As an alternative, passive immune therapeutics comprising antibodies have been used in clinically to directly induce apoptosis of tumor cells by ligating the targeted molecules (11), or act as antagonists of tumor growth factors (12). Moreover, these passive therapeutic antibodies trigger complement dependent cytotoxicity (CDC) or antibody dependent cellular cytotoxicity (ADCC) (12, 13), promote phagocytosis by dendritic cells (14), induce cross-talk among immune cells (NK and DCs), produce immunomodulatory cytokines (type I and type II interferons) (12), and enhance the cross-presentation of antigen presenting cells (APC) for the priming of CD8+ cytotoxic T lymphocytes (12, 14). By these reactions, passive therapeutic antibody drugs can be effective agents for tumor inhibition. The effectiveness of therapeutic antitumor antibodies portends the potential of enhanced or improved EAAs to function as effective therapeutic antitumor antibodies.

Recently, low-dose chemotherapy (metronomic chemotherapy) has been shown to induce an antitumor immune response and enhance the efficacy of cancer therapy. For example, the anti-microtubular taxanes (paclitaxel and docetaxel) were found to trigger the production of cytokines by macrophages to activate other immune cells such as dendritic cells (DC) (15), natural killer cells (NK) (16) and cytotoxic T lymphocytes (CTL) against tumors (16, 17). Paclitaxel also reduced the number of regulatory T cells (Treg) and myeloid derived suppressor cells (MDSC), and led to the augmentation of the functions of CD4 and CD8 T cells (16, 17). In other cases, DNA alkylating agents such as cyclophophamide and mafosfamide in low doses selectively depleted Treg cells (18, 19), caused an increase in Teff:Treg ratios via up-regulation of the Th17 pathway (20), and improved the outcome of tumor vaccinations against cancer (21-23). Furthermore, doxorubicin, mitomycin C, vinblastine and methotrexate in low doses all have been found to up-regulate DC maturation, antigen processing and antigen presentation, which led to synergistic antitumor effects of low dose chemotherapy combined with the DC vaccine (24-26). These various anti-tumor drugs in low doses can induce cell-mediated immunity against tumors, but they have less of a contribution to humoral immunity. Therefore, they have not been utilized to raise EAAs against tumor growth in patients.

P4N, a derivative of Nordihydroguaiaretic acid (NDGA), a natural product from the creosote bush, Larrea tridentate, is comprised of two phenolic rings connected by long and flexible —CH₂—CH₂— linkers to piperidines (27). Like NDGA and its methylated derivatives (28), P₄N has shown noteworthy antivirus and anticancer effects via competition with the transcription factor Sp1 for its DNA-binding site (29, 30). Although NDGA has been shown to inhibit 5-lipoxygenase, suppress the production of leukotriene B4, and activate macrophages, the immunoregulatory activity of P₄N is still unclear.

### SUMMARY OF THE INVENTION

In accordance with the present invention, the immunoregulatory activity of low doses of P₄N was investigated. Unlike previously described antitumor drugs, low dose P₄N, in doses of about 1 to 10 mg/kg, or at concentrations of about 10 to 100 nM, was surprisingly found to contribute to humoral immunity by raising the titers and activities of autoantibodies against GRP78 and F1F0 ATP synthase on the surface of CT26 cells, and inducing B cell proliferation and differentiation of plasma cells.

The present invention is the first to show that low concentrations of the antitumor drug, P₄N, contributes to the growth inhibition of tumors, such as colorectal tumors, by enhancing the production of endogenous autoantibodies. P₄N not only enhanced the proliferation of B cells which increased the titers of antibodies in sera (FIG. 3B), but it also increased the activities of anti-tumor autoantibodies (FIG. 15). FIG. 3F shows that although the titers of antitumor autoantibodies in PBS anti-sera, or P₄N anti-sera were different, they recognized the same antigens, GRP78 and F1F0 ATP synthase, in the membrane fraction (FIG. 3H), indicating that the better antitumor efficacy of P₄N anti-sera did not result from new antigen recognition, but it resulted instead from the improvement of the quantity and quality of the antitumor autoantibodies.

Therefore, in accordance with one or more embodiments, the present inventors now show that low dose P₄N induces B cell proliferation by activating LTA4H which enhances the production of leukotriene B4 and stimulates monocytes to release pro-inflammatory cytokines. Inflammatory monocytes then release Activin A as an autocrine signal to stimulate BAFF production via activation of the ALK4/Smad3 pathway and through BAFF, B cell proliferation and activation is enhanced.

In accordance with an embodiment, the present invention provides a method for inducing endogenous antitumor autoantibodies (EAA) in a subject having a neoplasia comprising administering to the subject an effective amount of the nordihydroguaiaretic acid (NDGA) derivative P₄N, having the following formula:

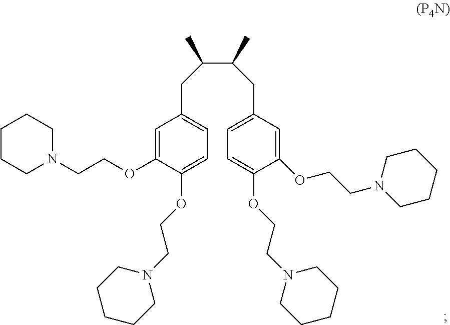 [SMILES: C[C@H](Cc1ccc(OCCN2CCCCC2)c(OCCN2CCCCC2)c1)[C@@H](C)Cc1ccc(OCCN2CCCCC2)c(OCCN2CCCCC2)c1]

or salts, solvates and stereoisomers thereof.

In accordance with another embodiment, the present invention provides a method for inducing B cell proliferation in a subject having a neoplasia comprising administering to the subject an effective amount of the nordihydroguaiaretic acid (NDGA) derivative P₄N, having the following formula:

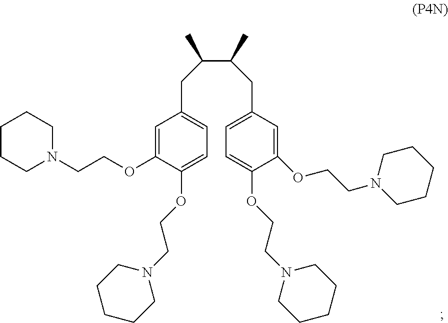 [SMILES: C[C@H](Cc1ccc(OCCN2CCCCC2)c(OCCN2CCCCC2)c1)[C@@H](C)Cc1ccc(OCCN2CCCCC2)c(OCCN2CCCCC2)c1]

or salts, solvates and stereoisomers thereof.

In accordance with a further embodiment, the present invention provides a method for inducing BAFF stimulated B cell proliferation in a subject having a neoplasia comprising administering to the subject an effective amount of the nordihydroguaiaretic acid (NDGA) derivative P₄N, having the following formula:

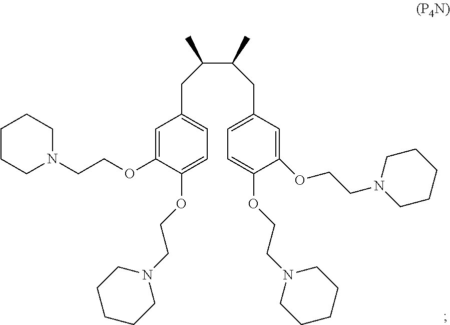 [SMILES: C[C@H](Cc1ccc(OCCN2CCCCC2)c(OCCN2CCCCC2)c1)[C@@H](C)Cc1ccc(OCCN2CCCCC2)c(OCCN2CCCCC2)c1]

or salts, solvates and stereoisomers thereof.

In accordance with still another embodiment, the present invention provides a method for suppressing or inhibition growth of a neoplasia in a subject having a neoplasia comprising administering to the subject an effective amount of a pharmaceutical composition comprising the nordihydroguaiaretic acid (NDGA) derivative P₄N, having the following formula:

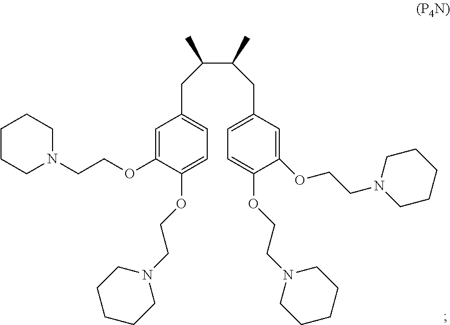 [SMILES: C[C@H](Cc1ccc(OCCN2CCCCC2)c(OCCN2CCCCC2)c1)[C@@H](C)Cc1ccc(OCCN2CCCCC2)c(OCCN2CCCCC2)c1]

or salts, solvates and stereoisomers thereof, and a pharmaceutically acceptable carrier.

### BRIEF DESCRIPTION OF THE DRAWINGS

FIGS. 1A-1C illustrate the antitumor effects of low dose P₄N in immune competent and deficient mice. (A) BALB/c (n=10/group) and nude mice (n=10/group) bearing CT26 tumors were treated with a single intratumoral injection of 2.5 or 5 mg/kg of P₄N N. (B) B cell or CD8+ T cell depleted BALB/c mice (n=9/group) bearing CT26 tumors were treated with 5 mg/kg of P4N by intratumoral injection every week. Tumor volumes were measured every 2 days after treatments. Significant differences between the P₄N groups and the PBS group were identified and labeled with *:p<0.05 and **:p<0.01. (C) Immunohistochemistry staining was used to monitor the infiltrations of macrophage (F4/80), DC (CD11c), T (CD3) and B cells (CD20) in the tumor area after a single intratumoral injection of PBS or 5 mg/kg of P₄N. The photos are representative of the mice sacrificed on day 7 after the treatments.

FIGS. 2A-2C show the effect of P₄N-induced anti-sera on the formation of pulmonary tumor nodules. (A) Metastatic CT26 tumor nodules in the lungs of mice with different treatments (n=5/group). (B) The number of lung metastases in each group were counted and were expressed as the mean±SD (n=5/group). (C) Mean survival rate of mice in each group (n=7/group) were calculated and plotted. Data were collected from two independent experiments. Significant differences between the P₄N anti-sera group and the PBS anti-sera group were determined and labeled with **P<0.01 or ***P<0.001.

FIGS. 3A-31 depict the antitumor effect of P₄N-induced anti-serum in immunodeficient mice and the target antigens of the anti-serum. (A) Nude mice bearing CT26 cell tumors (n=5/group) were treated with 100 μl of PBS, PBS anti-sera or P₄N anti-sera weekly. Tumor volumes were measured every 2 days after treatment. (B) The titers of specific anti-CT26 or JC cell antibodies for the PBS anti-sera or P₄N anti-sera on day 0, 17, 24 and 31 were measured. (C) The effects of P₄N on the changes in the isotypes of specific anti-CT26 cell antibodies were examined. The titers of these anti-sera (1600× dilution) were measured for IgM, IgG1, IgG2a, IgG2b or IgA. (D) Immunohistochemical analysis of anti-sera bound to tumor antigens on the surface of CT26. Color development by HRP-conjugated anti-mouse antibodies indicate the anti-sera antibody (E) Anti-sera binding to tumor antigens on the surface of CT26 cells was indirectly detected with FITC-conjugated goat anti-mouse IgG antibody. (F) The subcellular location of antigens recognized by the anti-sera was monitored by confocal microscopy. Alexa Fluor-568-conjugated anti-mouse Ig antibodies were used to display the presence of anti-sera (red). Alexa Fluor 488-ConA and DAPI were used to indicate the cell membrane (green) and the cell nucleus (blue), respectively. (G) The normal sera, PBS anti-sera or P₄N anti-sera were used to probe western blots of membrane proteins of extracted from CT26 cells. Two bands, protein a (78 kDa) and protein b (55 kDa) were visualized: E-cadherin proteins were used as a loading control. (H) Normal sera, PBS anti-sera or P₄N anti-sera were used in a co-immunoprecipitation assay. The co-precipitated proteins were analyzed by SDS-PAGE. Protein bands were visualized by Coomassie blue staining. Proteins a and b are indicated by an arrow. (I) Proteins a and b were analyzed by UPLC/HRMS/MS. Peptides identical to the partial protein sequences of GRP78 and ATP synthase are labeled in blue. For all results, significant differences between the P₄N anti-sera group and the PBS anti-sera group were calculated and labeled with *p<0.05.

FIG. 4 shows the gene expression profiles of P₄N treated THP-1 cells. cDNA microarrays probed with mRNA from untreated or P₄N-treated cells were analyzed and 26 up-regulated genes involved in cytokine-cytokine interaction and 10 up-regulated genes involved in cell proliferation highlighted (KEGG Pathway, Gene Ontology panels).

FIGS. 5A-5F depict the expression of Activin A/BAFF induced by P₄N treatments. (A) P₄N induced Activin A or BAFF expression in human PBMCs as determined by RT-PCR. S1-S5 display different individual blood samples collected from 5 different human donors; N, no treatment; P, P₄N-treatment (3 μM). The change in Activin A and BAFF expression (mean of 5 patients) relative to the control (NC) is plotted as a histogram (B) The effect of increasing dosages of P₄N for 12 h on mRNA and protein levels of Activin A or BAFF in THP-1 cells was analyzed by RT-PCR and ELISA. (C) The effect of 6 μM P₄N on mRNA and protein levels of Activin A or BAFF in THP-1 cells was determined at a different time period. (D) The effect of different inhibitors on Activin A-induced BAFF expression was examined. The expression of BAFF mRNA in inhibitor-treated THP-1 cells was determined by RT-PCR, and (E) BAFF protein secretion was measured by ELISA. (F) The effect of THP-1 conditioned media on the proliferation of Raji cells and OKT3 hybridoma cells were determined. Media were harvested, in which THP-1 cells were pre-treated without inhibitor (2) or with SB431542 (3), A83-01 (4), SIS3 (5), SB203580 (6) or PD98059 (7) and then treated with P₄N for 12 h. The results are expressed as % viable cells relative to the control (NC, media from untreated THP-1 cells). All values are the means of triplicate samples±SEM. The significant differences in the results of inhibitor and P₄N-treated groups compared with the untreated group are indicated by ***P<0.001.

FIGS. 6A-6E show P₄N directly regulated LTA4H to induce monocyte inflammation. (A) P₄N (orange) was docked with LTA4H by iGEMDOCK software (gemdock.life.nctu.edu.tw/dock/igemdock.php). RB3041 (Pink) is a small molecule inhibitor that identifies the active site of LTA4H. The amino acids that potentially interact with P₄N are noted. The effects of P₄N and bestatin on monocytes and their expression of (B) LTB4, (C) TNF-α, (D) IL-8, or (E) Activin A were determined and shown for different times. All values were expressed as the mean±SEM. **P<0.05, group P₄N vs group (P₄N+bestatin).

FIG. 7 depicts the expression of TNF-α and IL-8 in the tumors. Immunohistochemistry staining was used to monitor the expression of TNF-α and IL-8 in the tumor area after a single intratumoral injection of PBS or 5 mg/kg of P₄N. The photos are representative of the mice sacrificed on day 7 after the treatments.

FIG. 8 shows the effect of PBS anti-sera and P₄N anti-sera on cell proliferation in CT-26 cells. The proliferation of CT-26 cells treated with 1, 2, 4 or 8 μl sera was analyzed by MTT assay. De-anti-CT26 Abs sera: the anti-CT-26 autoantibodies from P₄N anti-sera were pre-subtracted by CT-26 cells binding. The data are reported as the proliferation index. Significant differences between the P₄N-treated groups and the untreated group were indicated by *(p<0.05) (n=5).

FIGS. 9A-9F show the effect of P₄N on B cell activation. (A) The proliferation of human PBMCs treated with different doses P₄N was analyzed by MTT assay. The data are reported as the proliferation index. Significant differences between the P₄N-treated groups and the untreated group were indicated by *(p<0.05) (n=4). (B) The effect of P4N on B cell proliferation was determined by flow cytometry. PBMCs from six healthy individuals were examined, and the results displayed with different colors. (C) The effects of P4N on the changes in total B, naïve B or activated B cells among mouse splenocytes in vitro. Significant differences in the results of P₄N treatments compared with untreated results are indicated by **(p<0.01) (n=3). (D) The effect of P₄N treatment on specific antibody production in antigen-immunized mice was monitored. The titers of total specific EGFP Ig's in sera were measured by ELISA (n=5). (E) The effect of P₄N treatment on the changes in antibody classes was examined (n=5). (F) The changes in B cell types after antigen immunization with P₄N. All values (n=3) were expressed as mean±SEM at the same dilutions. The significant differences in the results of P4N-treated groups compared with the untreated group are indicated by *P<0.05, **P<0.01 or ***P<0.001.

FIG. 10 depicts the effects of P₄N on cell proliferation. Human B or T cells were isolated from the PBMCs by negative selection using a magnetic sorting device (Miltenyi Biotec, Auburn, Calif.). Cell proliferation was determined by an MTT assay.

FIG. 11 shows cytokine and chemokine profiles of P₄Ntreated PBMC. PBMCs isolated from 7 healthy donors were treated with 3 μM of P₄N for 48 h. Supernatants were collected and analyzed for human cytokines using the Bio-Plex assay. Shown are the changed results of the production of 27 human cytokines when PBMCs were treated with P₄N in three independent experiments. The levels of IL-4, IL-5, IL-7, IL-9, Eotaxin and FGF were undetectable.

FIG. 12 shows cytokine and chemokine profiles of P₄N treated THP-1. THP-1 cells were treated with 3 μM of P₄N or the vehicle control (NC) for 24 h. Supernatants were collected and analyzed using the Bio-Plex assay for human cytokines. Shown are the concentrations of 27 human cytokines for each treatment from three independent experiments. The levels of IL-2, IL-4, IL-6, IL-7, IL-9, IL13, IL-15, Eotaxin, G-CSF, IFN-γ and PDGF-bb were undetectable.

FIG. 13 provides graphs showing P₄N-induced pro-inflammatory cytokine production in THP-1 cells. THP-1 cells were treated with 3 μM P₄N for different lengths of time, Cell supernatants were collected and TNF-α, IL-1β and IL-8 quantitated by ELISA. All results are the mean of three independent experiments (n=6).

FIGS. 14A-14C depict P₄N-induced mouse B cell proliferation via the Activin A/BAFF pathway. (A) The proliferation of P₄N-treated splenocytes. Splenocytes isolated from three mice were individually treated with increasing concentrations of P₄N for 48 h and their proliferation was analyzed by MTT assay. The data are reported as the proliferation index. The results that differ significantly from the untreated group are indicated by *(p<0.05) (n=6). (B) The proliferation of B cells in mouse splenocytes treated with low doses of P₄N. After treatment with P₄N for 48 h, B-cells were identified by CD19 surface marker staining and the cell number was calculated following a flow cytometer analysis. The results that differ significantly from the untreated group are indicated by *(p<0.05) (n=6).(C) P₄N enhanced Activin A and BAFF expression in mouse splenocytes. Levels of Activin A and BAFF transcripts were measured by RT-PCR. Each sample obtained from 3 different mice; N, no treatment; P, P₄N-treatment.

FIG. 15 is a graph showing the affinities of anti-sera against CT-26 cells after different lengths of incubation. Tumor antigens on the surface of CT26 cells were indirectly probed with P₄N and PBS anti-sera and FITC-conjugated goat anti-mouse IgG antibody. The specific fluorescent activities were measured by flow cytometry.

### DETAILED DESCRIPTION OF THE INVENTION

In accordance with an embodiment, the present invention provides the present invention provides a method for inducing endogenous antitumor autoantibodies (EAA) in a subject having a neoplasia comprising administering to the subject an effective amount of a nordihydroguaiaretic acid (NDGA) derivative of formula I:

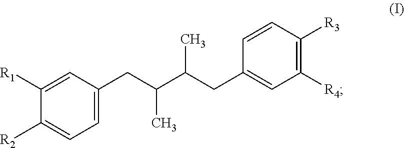

wherein R₁, R₂, R₃ and R₄ independently represent hydroxy, a straight or branched chain lower alkyl or alkoxy, an amino acid residue, a substituted amino acid residue, a nitrogen-containing 5- or 6-membered heterocyclic ring or a saccharide residue; the amino acid residue, substituted amino acid residue, nitrogen-containing 5 or 6 membered heterocyclic ring or saccharide residue being optionally joined to the phenyl ring by a linker of an oxygen atom and 1-10 carbon atoms.

In accordance with an embodiment, the derivative of NDGA used in the methods of the present invention is where R₁, R₂, R₃ and R₄ each are 2-(piperidino)ethoxyphenyl groups. The compound P₄N (tetrapiperidino NDGA, meso-2,3-dimethyl-1,4-bis(3,4-[2-(piperidino)ethoxyphenyl])butane tetrakishydrochloride salt) has the following formula:

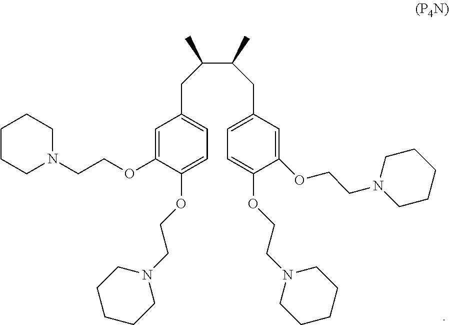 [SMILES: C[C@H](Cc1ccc(OCCN2CCCCC2)c(OCCN2CCCCC2)c1)[C@@H](C)Cc1ccc(OCCN2CCCCC2)c(OCCN2CCCCC2)c1]

The synthesis of P4N is detailed in U.S. Pat. No. 7,741,357, issued Jun. 22, 2010 and incorporated by reference herein.

In accordance with an embodiment, the present invention provides a method for inducing endogenous antitumor autoantibodies (EAA) in a subject having a neoplasia comprising administering to the subject an effective amount of the nordihydroguaiaretic acid (NDGA) derivative P₄N, having the following formula:

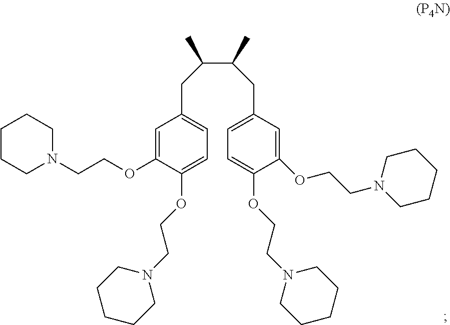 [SMILES: C[C@H](Cc1ccc(OCCN2CCCCC2)c(OCCN2CCCCC2)c1)[C@@H](C)Cc1ccc(OCCN2CCCCC2)c(OCCN2CCCCC2)c1]

or salts, solvates and stereoisomers thereof.

In accordance with another embodiment, the present invention provides a method for inducing B cell proliferation in a subject having a neoplasia comprising administering to the subject an effective amount of the nordihydroguaiaretic acid (NDGA) derivative P₄N, having the following formula:

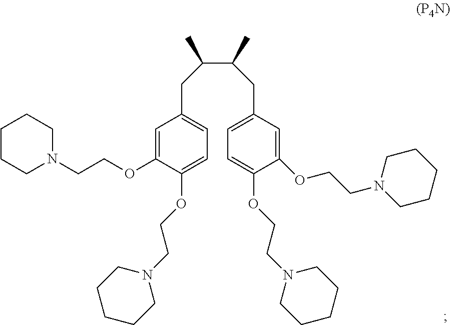 [SMILES: C[C@H](Cc1ccc(OCCN2CCCCC2)c(OCCN2CCCCC2)c1)[C@@H](C)Cc1ccc(OCCN2CCCCC2)c(OCCN2CCCCC2)c1]

or salts, solvates and stereoisomers thereof.

In accordance with a further embodiment, the present invention provides a method for inducing BAFF stimulated B cell proliferation in a subject having a neoplasia comprising administering to the subject an effective amount of the nordihydroguaiaretic acid (NDGA) derivative P₄N, having the following formula:

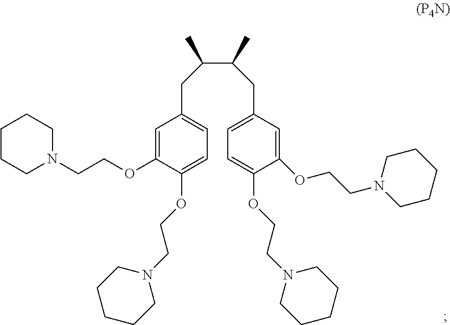 [SMILES: C[C@H](Cc1ccc(OCCN2CCCCC2)c(OCCN2CCCCC2)c1)[C@@H](C)Cc1ccc(OCCN2CCCCC2)c(OCCN2CCCCC2)c1]

or salts, solvates and stereoisomers thereof.

In accordance with still another embodiment, the present invention provides a method for suppressing or inhibition growth of a neoplasia in a subject having a neoplasia comprising administering to the subject an effective amount of a pharmaceutical composition comprising the nordihydroguaiaretic acid (NDGA) derivative P₄N, having the following formula:

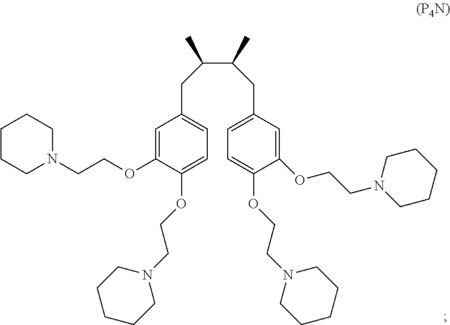 [SMILES: C[C@H](Cc1ccc(OCCN2CCCCC2)c(OCCN2CCCCC2)c1)[C@@H](C)Cc1ccc(OCCN2CCCCC2)c(OCCN2CCCCC2)c1]

or salts, solvates and stereoisomers thereof, and a pharmaceutically acceptable carrier.

Included within the compounds of the present invention are the tautomeric forms of the disclosed compounds, isomeric forms including diastereoisomers, and the pharmaceutically-acceptable salts thereof. The term “pharmaceutically acceptable salts” embraces salts commonly used to form alkali metal salts and to form addition salts of free acids or free bases. Examples of acids which may be employed to form pharmaceutically acceptable acid addition salts include such inorganic acids as hydrochloric acid, sulphuric acid and phosphoric acid, and such organic acids as maleic acid, succinic acid and citric acid. Other pharmaceutically acceptable salts include salts with alkali metals or alkaline earth metals, such as sodium, potassium, calcium and magnesium, or with organic bases, such as dicyclohexylamine. Suitable pharmaceutically acceptable salts of the compounds of the present invention include, for example, acid addition salts which may, for example, be formed by mixing a solution of the compound according to the invention with a solution of a pharmaceutically acceptable acid, such as hydrochloric acid, sulphuric acid, methanesulphonic acid, fumaric acid, maleic acid, succinic acid, acetic acid, benzoic acid, oxalic acid, citric acid, tartaric acid, carbonic acid or phosphoric acid. All of these salts may be prepared by conventional means by reacting, for example, the appropriate acid or base with the corresponding compounds of the present invention.

Salts formed from free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

For use in medicines, the salts of the compounds of the present invention should be pharmaceutically acceptable salts. Other salts may, however, be useful in the preparation of the compounds according to the invention or of their pharmaceutically acceptable salts.

In addition, embodiments of the invention include hydrates of the compounds of the present invention. The term “hydrate” includes but is not limited to hemihydrate, monohydrate, dihydrate, trihydrate and the like. Hydrates of the compounds of the present invention may be prepared by contacting the compounds with water under suitable conditions to produce the hydrate of choice.

With respect to the pharmaceutical compositions described herein, the carrier can be any of those conventionally used, and is limited only by physico-chemical considerations, such as solubility and lack of reactivity with the active compound(s), and by the route of administration. The carriers described herein, for example, vehicles, adjuvants, excipients, and diluents, are well-known to those skilled in the art and are readily available to the public. It is preferred that the carrier be one which is chemically inert to the active agent(s), and one which has little or no detrimental side effects or toxicity under the conditions of use. Examples of the carriers include solid compositions such as solid-state carriers or latex beads.

Solid carriers or diluents include, but are not limited to, gums, starches (e.g., corn starch, pregelatinized starch), sugars (e.g., lactose, mannitol, sucrose, dextrose), cellulosic materials (e.g., microcrystalline cellulose), acrylates (e.g., polymethylacrylate), calcium carbonate, magnesium oxide, talc, or mixtures thereof.

The choice of carrier will be determined, in part, by the particular pharmaceutical composition, as well as by the particular method used to administer the composition. Accordingly, there are a variety of suitable formulations of the pharmaceutical composition of the invention.

In accordance with an embodiment, the present invention provides a use of the pharmaceutical compositions disclosed herein in an amount effective for use in a medicament, and most preferably for use as a medicament for treating a disease or disorder associated with a neoplastic disease in a subject.

The compound and compositions used in the methods of the present invention can be used to treat a variety of tumors and cancers, including, without limitation, hematological malignancies such as acute lymphoblastic leukemia, acute myeloid leukemia, chronic lymphocytic leukemia, chronic myelogenous leukemia, childhood acute leukemia, non-Hodgkin's lymphoma, chronic lymphocytic leukemia, malignant cutaneous T-cells, mycosis fungoides, non-MF cutaneous T-cell lymphoma, lymphomatoid papulosis, T-cell rich cutaneous lymphoid hyperplasia, bullous pemphigoid, discoid lupus erythematosus, lichen planus, adrenocortical carcinoma, anal cancer, astrocytoma, bile duct cancer, bladder cancer, bone cancer osteosarcoma/malignant fibrous histiocytoma, neurological malignancies such as neuroblastoma, glioblastoma, astrocytoma, gliomas, brain stem glioma, brain tumor ependymoma, brain tumor medulloblastoma, neuroblastoma glioblastoma, breast cancer, carcinoid tumor gastrointestinal, carcinoma adrenocortical, carcinoma islet cell, cervical cancer, clear cell sarcoma of tendon sheaths, colon cancer, cutaneous T-cell lymphoma, endometrial cancer, epithelial cancer ovarian, esophageal cancer, Ewing's family of tumors, extragonadal germ cell tumor, extrahepatic bile duct cancer, eye cancer, intraocular melanoma, ductal cancer, eye cancer retinoblastoma, dysplastic oral mucosa, invasive oral tumor, gallbladder cancer, gastric (stomach) cancer, gastrointestinal carcinoid tumor, germ cell tumor extragonadal, germ cell tumor, ovarian tumor, gestational trophoblastic tumor, glioblastoma, glioma, hairy cell leukemia, hepatocellular (liver) cancer, Hodgkin's lymphoma, hypopharyngeal cancer, intraocular melanoma, islet cell carcinoma (endocrine pancreas), Kaposi's sarcoma, laryngeal cancer, leukemia acute lymphoblastic cancer, leukemia acute myeloid cancer, leukemia chronic lymphocytic cancer, leukemia chronic myelogenous cancer, leukemia hairy cell cancer, liver cancer, non-small cell lung cancer, small cell lung cancer, male breast cancer, malignant mesothelioma, medulloblastoma, melanoma, merkel cell carcinoma, multiple endocrine neoplasia syndrome, mycosis fungoides, myeloma multiple, nasal cavity, paranasal and sinus cancer, nasopharyngeal cancer, neuroblastoma, oral cavity and lip cancer, oropharyngeal cancer, osteosarcoma/malignant fibrous histiocytoma of bone, ovarian epithelial cancer, ovarian germ cell tumor, pancreatic cancer, parathyroid cancer, penile cancer, pheochromocytoma, pineal and supratentorial primitive neuroectodermal tumors, pituitary tumor, pleuropulmonary blastoma, prostate cancer, rectal cancer, renal, pelvis and ureter transitional cell cancer, retinoblastoma, rhabdomyosarcoma, salivary gland cancer, sarcoma soft tissue adult, Sezary syndrome, skin cancer, small intestine cancer, stomach (gastric) cancer, testicular cancer, thymoma, thyroid cancer, urethral cancer, transitional and squamous cell urinary carcinoma, gynecological cancer such as cervical cancer ovarian cancer, uterine cancer, endometrial cancer, vaginal cancer, vulvar cancer, Waldenstrom's macroglobulinemia, and Wilms' tumor, testicular tumors; liver tumors including hepatocellular carcinoma (“HCC”) and tumor of the biliary duct; multiple myelomas; tumors of the esophageal tract; other lung tumors including small cell and clear cell; Hodgkin's lymphomas; sarcomas in different organs; as well as those mentioned above, as well as micro and macro metastases, and the like.

As used herein, the term “micrometastases” means a small collection of cancer cells that have been shed from the original tumor and spread to another part of the body. They cannot be seen with any imaging tests such as a mammogram, MRI, ultrasound, PET, or CT scans.

As used herein, the term “macrometastases” means a larger collection of cancer cells that have been shed from the original tumor and spread to another part of the body, and are capable of being detected either visually, or with the aid of any imaging tests such as a mammogram, MRI, ultrasound, PET, or CT scans.

In a preferred embodiment, the methods of the present invention can be used to treat colorectal cancer.

It will be understood to those of skill in the art that the term “therapeutic agent” is any agent capable of affecting the structure or function of the body of a subject or is an agent useful for the treatment or modulation of a disease or condition in a subject suffering therefrom. Examples of therapeutic agents can include any drugs known in the art for treatment of disease indications.

An active agent and a biologically active agent are used interchangeably herein to refer to a chemical or biological compound that induces a desired pharmacological and/or physiological effect, wherein the effect may be prophylactic or therapeutic. The terms also encompass pharmaceutically acceptable, pharmacologically active derivatives of those active agents specifically mentioned herein, including, but not limited to, salts, esters, amides, prodrugs, active metabolites, analogs and the like. When the terms “active agent,” “pharmacologically active agent” and “drug” are used, then, it is to be understood that the invention includes the active agent per se, as well as pharmaceutically acceptable, pharmacologically active salts, esters, amides, prodrugs, metabolites, analogs etc.

Specific examples of useful biologically active agents the above categories include: anti-neoplastics such as androgen inhibitors, antimetabolites, cytotoxic agents, and immunomodulators. Antineoplastic agents can include, for example, alkylating agents, nitrogen mustard alkylating agents, nitrosourea alkylating agents, antimetabolites, purine analog antimetabolites, pyrimidine analog antimetabolites, hormonal antineoplastics, natural antineoplastics, antibiotic natural antineoplastics, and vinca alkaloid natural antineoplastics. Further examples can include alkylating antineoplastic agents, such as carboplatin and cisplatin; nitrosourea alkylating antineoplastic agents, such as carmustine (BCNU); antimetabolite antineoplastic agents, such as methotrexate; pyrimidine analog antineoplastic agents, such as fluorouracil (5-FU) and gemcitabine; hormonal antineoplastics, such as goserelin, leuprolide, and tamoxifen; natural antineoplastics, such as aldesleukin, interleukin-2, docetaxel, etoposide, interferon; paclitaxel, other taxane derivatives, and tretinoin (ATRA); antibiotic natural antineoplastics, such as bleomycin, dactinomycin, daunorubicin, doxorubicin, and mitomycin; and vinca alkaloid natural antineoplastics, such as vinblastine and vincristine.

In a further embodiment, the compositions and methods of the present invention can be used in combination with one or more additional therapeutically active agents which are known to be capable of treating conditions or diseases discussed above. For example, the compositions of the present invention could be used in combination with one or more known therapeutically active agents, to treat a proliferative disease such as a tumor or cancer. Non-limiting examples of other therapeutically active agents that can be readily combined in a pharmaceutical composition with the compositions and methods of the present invention are enzymatic nucleic acid molecules, allosteric nucleic acid molecules, antisense, decoy, or aptamer nucleic acid molecules, antibodies such as monoclonal antibodies, small molecules, and other organic and/or inorganic compounds including metals, salts and ions.

As used herein, the term “subject” refers to any living mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

The dose of the compositions of the present invention also will be determined by the existence, nature and extent of any adverse side effects that might accompany the administration of a particular composition. Typically, an attending physician will decide the dosage of the pharmaceutical composition with which to treat each individual subject, taking into consideration a variety of factors, such as age, body weight, general health, diet, sex, compound to be administered, route of administration, and the severity of the condition being treated.

In accordance with an embodiment of the present invention, it was found that use of low doses of P₄N, dose that were not cytotoxic to cancer cells directly in animal or cell based assays, could induce EAA and suppress tumor growth in a subject. By way of example, and not intending to limit the invention, the dose of the pharmaceutical compositions of the present invention can be about 0.1 to about 10 mg/kg body weight of the subject being treated, from about 1 to about 8 mg/kg body weight, from about 2.5 mg/kg to about 10 mg/kg, and from about 0.5 mg to about 5 mg/kg body weight.

The dose of the compositions of the present invention also will be determined by the existence, nature and extent of any adverse side effects that might accompany the administration of a particular composition. Typically, an attending physician will decide the dosage of the pharmaceutical composition with which to treat each individual subject, taking into consideration a variety of factors, such as age, body weight, general health, diet, sex, compound to be administered, route of administration, and the severity of the condition being treated.

As used herein, the terms “effective amount” or “sufficient amount” are equivalent phrases which refer to the amount of a therapy (e.g., a prophylactic or therapeutic agent), which is sufficient to induce EAA to a neoplasia in a subject and thereby reduce the severity and/or duration of a disease, ameliorate one or more symptoms thereof, prevent the advancement of a disease or cause regression of a disease, or which is sufficient to result in the prevention of the development, recurrence, onset, or progression of a disease or one or more symptoms thereof, or enhance or improve the prophylactic and/or therapeutic effect(s) of another therapy (e.g., another therapeutic agent) useful for treating a disease, such as a neoplastic disease or tumor.

Pharmaceutical compositions in accordance with the invention are useful for diagnosis, prognosis, prophylaxis or treatment of a condition. Accordingly, compositions in accordance with the invention are useful as a drug or as information for structural modification of existing compounds, e.g., by rational drug design. Compounds and methods of the invention are useful for screening compounds having an effect on a variety of conditions.

For therapeutic uses, the compositions or agents identified using the methods disclosed herein may be administered systemically, for example, formulated in a pharmaceutically-acceptable buffer such as physiological saline. Preferable routes of administration include, for example, subcutaneous, intravenous, intraperitoneally, intramuscular, or intradermal injections that provide continuous, sustained levels of the drug in the patient. Treatment of human patients or other animals are generally carried out using a therapeutically effective amount of a therapeutic of the invention in a physiologically-acceptable carrier. Suitable carriers and their formulation are described, for example, in Remington's Pharmaceutical Sciences by E. W. Martin.

The amount of the therapeutic agent to be administered varies depending upon the manner of administration, the age and body weight of the subject/patient, and with the subject's symptoms and condition. A compound is administered at a dosage that best achieves medical goals with the fewest corresponding side effects.

The pharmaceutical compositions of this invention including biologically active fragments, variants, or analogs thereof, can be administered by any suitable routes including intracranial, intracerebral, intraventricular, intrathecal, intraspinal, oral, topical, rectal, transdermal, subcutaneous, intravenous, intramuscular, intranasal, and the like. In one embodiment, the compositions are added to a retained physiological fluid, such as cerebrospinal fluid, blood, or synovial fluid. The compositions of the invention can be amenable to direct injection or infusion at a site of disease or injury.

As noted above, compositions of the invention can be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, cited herein.

For example, pharmaceutical compositions according to the invention may be in the form suitable for sterile injection. To prepare such a composition, the compositions(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate).

Suitable dosage forms can be formulated for, but are not limited to, oral, rectal, sublingual, mucosal, nasal, ophthalmic, subcutaneous, intramuscular, intravenous, transdermal, spinal, intrathecal, intra-articular, intra-arterial, sub-arachinoid, bronchial, lymphatic, and intra-uterille administration, and other dosage forms for systemic delivery of active ingredients. In a preferred embodiment, the dosage form is suitable for injection or intravenous administration.

To prepare such pharmaceutical dosage forms, one or more of the aforementioned compounds are intimately admixed with a pharmaceutical carrier according to conventional pharmaceutical compounding techniques. The carrier may take a wide variety of forms depending on the form of preparation desired for administration.

For parenteral formulations, the carrier will usually comprise sterile water, though other ingredients, for example, ingredients that aid solubility or for preservation, may be included. Injectable solutions may also be prepared in which case appropriate stabilizing agents may be employed.

In preparing the compositions in oral dosage form, any of the usual pharmaceutical media may be employed. Thus, for liquid oral preparations, such as, for example, suspensions, elixirs and solutions, suitable carriers and additives include water, glycols, oils, alcohols, flavoring agents, preservatives, coloring agents and the like. For solid oral preparations such as, for example, powders, capsules and tablets, suitable carriers and additives include starches, sugars, diluents, granulating agents, lubricants, binders, disintegrating agents and the like. Due to their ease in administration, tablets and capsules represent the most advantageous oral dosage unit form. If desired, tablets may be sugar coated or enteric coated by standard techniques.

Compositions for parenteral use may be provided in unit dosage forms (e.g., in single-dose ampules), or in vials containing several doses and in which a suitable preservative may be added. The composition may be in the form of a solution, a suspension, an emulsion, an infusion device, or a delivery device for implantation, or it may be presented as a dry powder to be reconstituted with water or another suitable vehicle before use. The composition may include suitable parenterally acceptable carriers and/or excipients.

In one approach, a therapeutic of the invention is provided within an implant, such as an osmotic pump, or in a graft comprising appropriately transformed cells. Methods of introduction may also be provided by rechargeable or biodegradable devices. Various slow release polymeric devices have been developed and tested for the controlled delivery of drugs, including proteinacious biopharmaceuticals. A variety of biocompatible polymers (including hydrogels), including both biodegradable and non-degradable polymers, can be used to form an implant for the sustained release of a bioactive factor at a particular target site.

Generally, the amount of administered agent of the invention (dosage) will be empirically determined in accordance with information and protocols known in the art.

Compositions of the invention can comprise various pharmaceutically acceptable salts, ether derivatives, ester derivatives, acid derivatives, and aqueous solubility altering derivatives of the active compound. The present invention can comprise all individual enantiomers, diastereomers, racemates, and other isomer of compounds of the invention. The invention also includes all polymorphs and solvates, such as hydrates and those formed with organic solvents, of this compound. Such isomers, polymorphs, and solvates may be prepared by methods known in the art, such as by regiospecific and/or enantioselective synthesis and resolution, based on the disclosure provided herein.

Suitable salts of the compound include, but are not limited to, acid addition salts, such as those made with hydrochloric, hydrobromic, hydroiodic, perchloric, sulfuric, nitric, phosphoric, acetic, propionic, glycolic, lactic pyruvic, malonic, succinic, maleic, fumaric, malic, tartaric, citric, benzoic, carbonic cinnamic, mandelic, methanesulfonic, ethanesulfonic, hydroxyethanesulfonic, benezenesulfonic, p-toluene sulfonic, cyclohexanesulfamic, salicyclic, p-aminosalicylic, 2-phenoxybenzoic, and 2-acetoxybenzoic acid; salts made with saccharin; alkali metal salts, such as sodium and potassium salts; alkaline earth metal salts, such as calcium and magnesium salts; and salts formed with organic or inorganic ligands, such as quaternary ammonium salts.

Additional suitable salts include, but are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate,

hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate salts of the compound of the present invention.

Prodrugs and active metabolites of compounds of the invention are also within the scope of the invention.

A prodrug is a pharmacologically inactive compound that is converted into a pharmacologically active agent by a metabolic transformation. In vivo, a prodrug is acted on by naturally occurring enzyme(s) resulting in liberation of the pharmacologically active agent. Conventional procedures for the selection and preparation of suitable prodrug derivatives are described, for example, in “Design of Prodrugs”, ed. H. Bundgaard, Elsevier, 1985.

An active metabolite is a compound which results from metabolism of another compound after administration of the latter to a subject. Metabolites can be identified by techniques well-known in the art.

It is also contemplated that in an embodiment of the present invention, the methods of treatment disclosed herein are useful against many mammalian tumors, including, for example, breast cancer, prostate cancer, pancreatic cancer, colon cancer, hepatoma, glioblastoma, ovarian cancer, leukemia, Hodgkin's lymphoma and multiple myeloma.

It will be understood by those of ordinary skill in the art that the term “tumor” as used herein means a neoplastic growth which may, or may not be malignant. Additionally, the compositions and methods provided herein are not only useful in the treatment of tumors, but in their micrometastses and their macrometastses. Typically, micrometastasis is a form of metastasis (the spread of a cancer from its original location to other sites in the body) in which the newly formed tumors are identified only by histologic examination; micrometastases are detectable by neither physical exam nor imaging techniques. In contrast, macrometastses are usually large secondary tumors.

In accordance with an embodiment, the present invention provides compositions and methods for the prevention and/or treatment of tumors, and their micrometastses and their macrometastses.

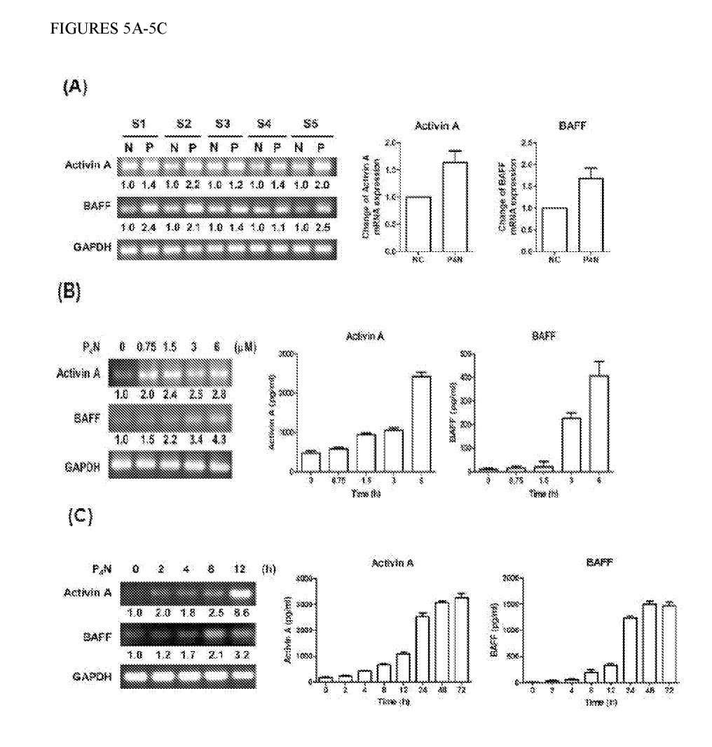

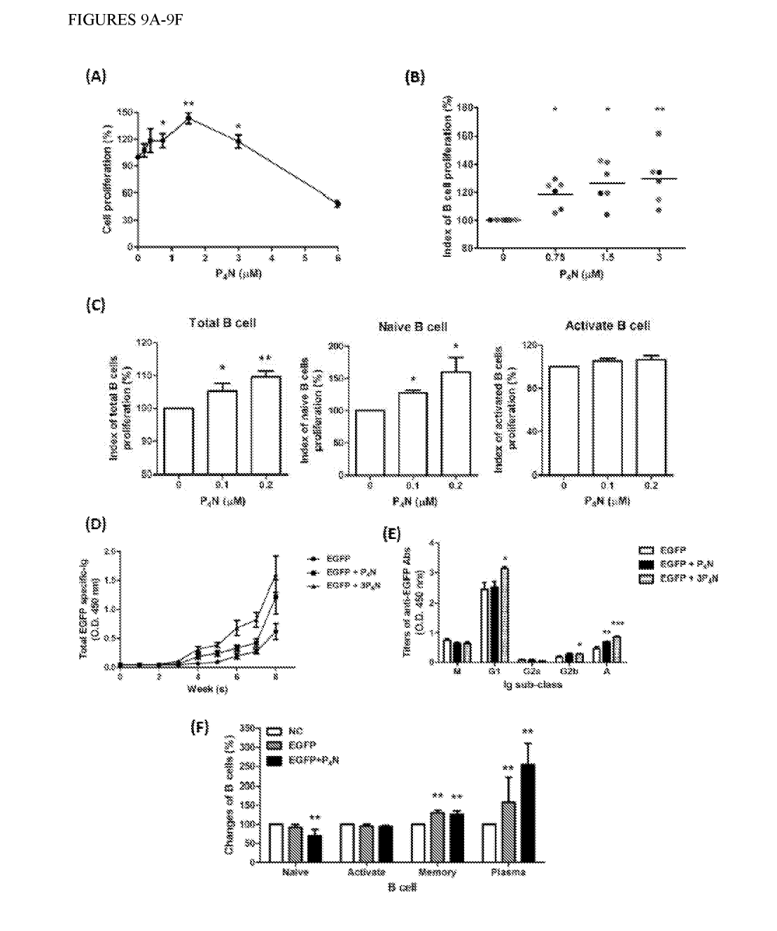

Without stimulation of antigen, B cells can't be activated and differentiate into plasma cells. In such condition, P₄N causes B cell proliferation and these proliferative B cells will arrest at the state of naïve B cell (FIGS. 9B and C). With stimulation of antigen and the help of CD4+T cell, these proliferative B cells by P₄N will be activated and differentiating into plasma cells (FIG. 9F). Thus, the inventors have shown that the promotion of EAA production by P₄N treatments is due to P₄N-accelerated B cell proliferation and differentiation to plasma cells (FIG. 9F). P₄N-induced B cell proliferation requires BAFF expression (FIG. 5), which is induced by Activin A through the ALK4/Smad3 signaling pathway. Similarly, Kim et al. found that Activin A can increase BAFF promoter activity and transcription via the ALK4-Smad3 pathway in mice APCs (31). The finding that P₄N-induced Activin A is involved in the stimulation of B cell proliferation is in accord with the report by Ogawa K. and co-workers that shows that LPS-induced Activin A can directly or indirectly increase B cell proliferation and Ig production (34). Other reports have shown that Activin A induces growth arrest and apoptosis in B cell-derived cells and hybridomas (35, 36). Activin A-induced apoptosis of B cells relies on the regulation of Bcl-family gene expression (37, 38). Although Activin A may cause B cell apoptosis, BAFF can up-regulate the expression of Bcl-2a1 and down-regulate the expression of BIM for B cell survival (39). Our data indicates that, P₄N-induced B cell proliferation is the result of BAFF directly stimulating the growth of B cells and preventing Activin A-induced B cell apoptosis.

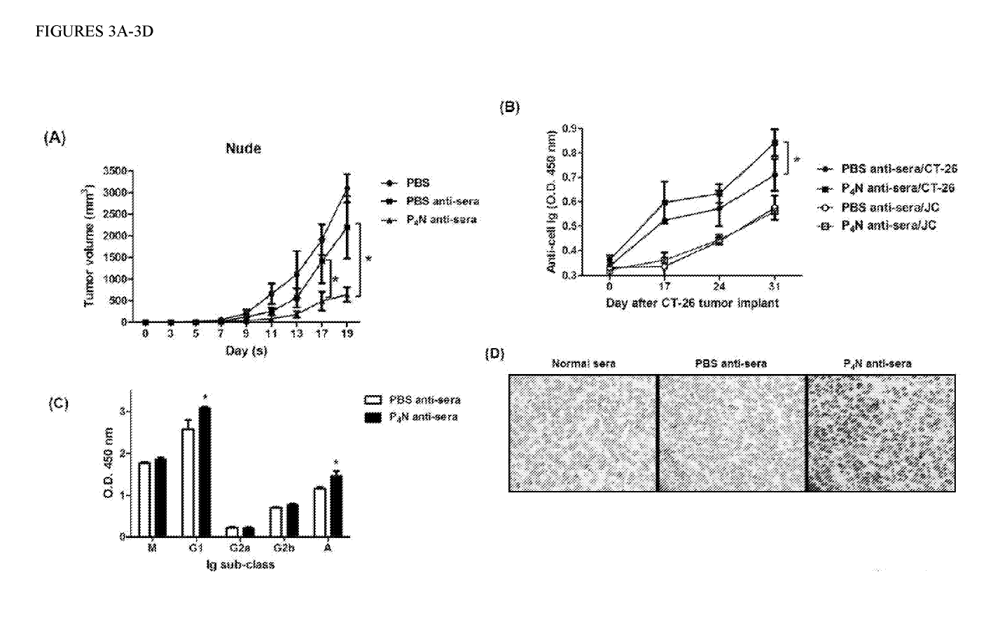

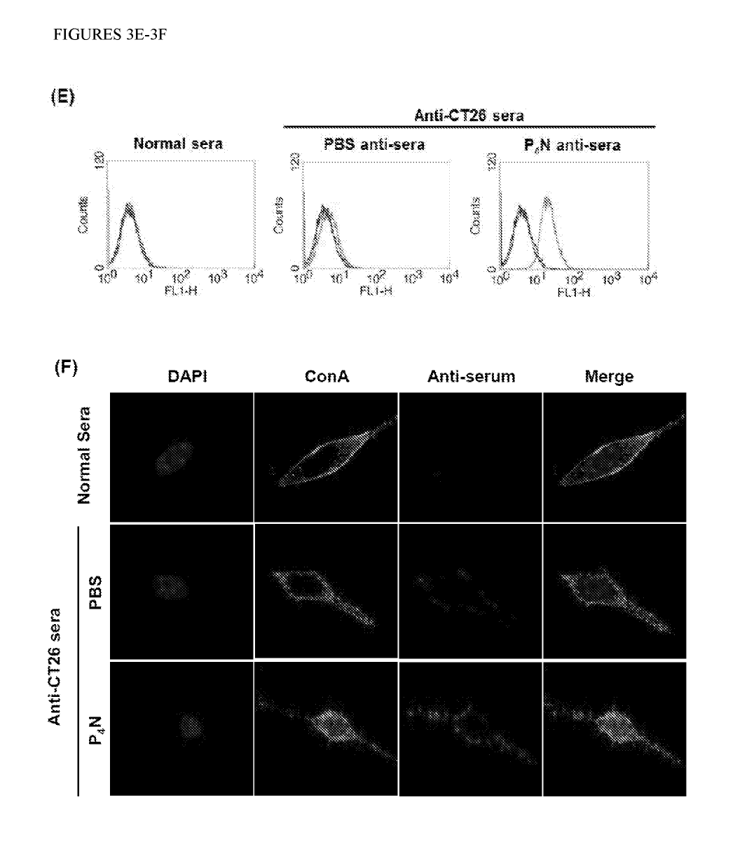

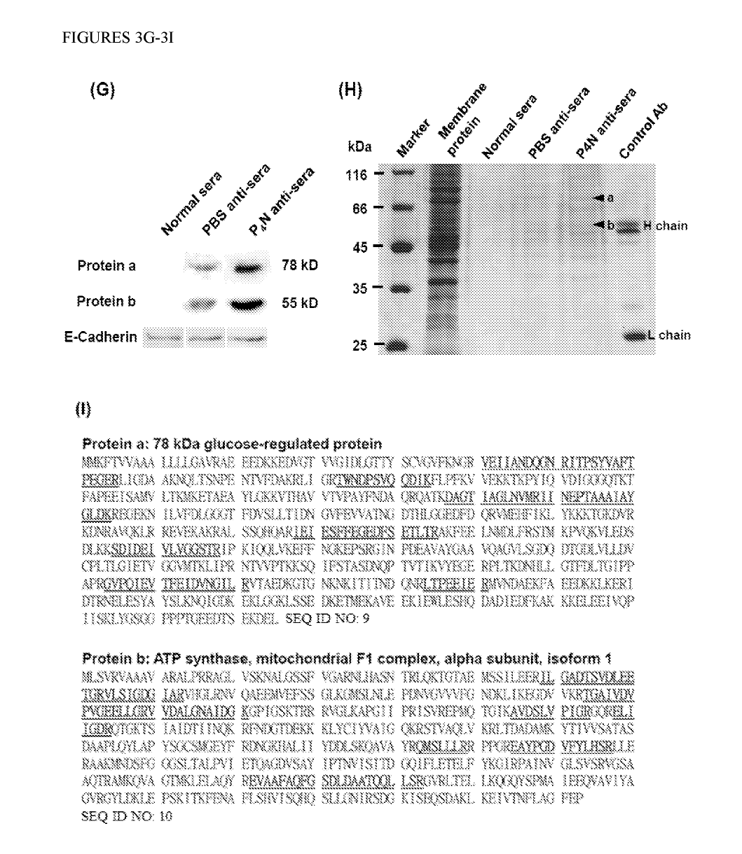

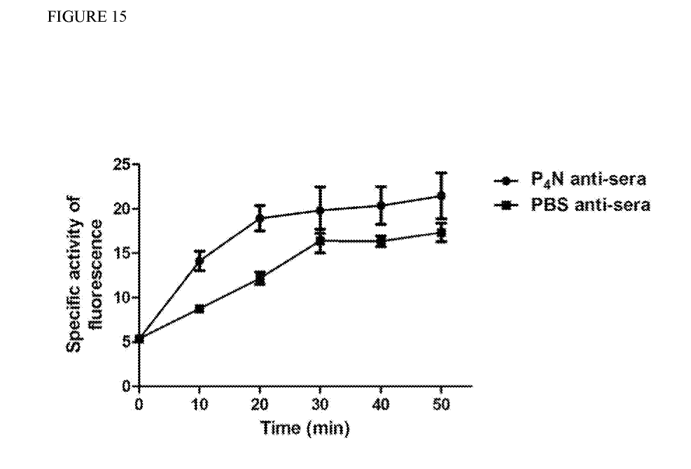

P₄N improved the quality of the EAA response by inducing Ig class switching from IgM to IgG1 and IgA (FIG. 3C and FIG. 9E), and it enhanced their binding affinities (FIG. 15). In the body, IgG1 and IgA easily diffuse into extravascular sites to access antigens, and have a greater efficacy for activating the complement system. Moreover, IgG1 also has a longer half-life in the blood and can efficiently sensitize NK cells for killing. Thus, the P₄N induced class-switch may be one of the reasons that the P₄N anti-sera cause more efficient suppression of tumor growth than PBS anti-sera. Another reason may be that P₄N enhances the process of somatic hypermutation in B cells that causes the antitumor autoantibodies in P₄N anti-sera to have higher antigen binding affinities than those in PBS anti-sera, which results in the antitumor autoantibodies being able to stably bind on the tumor surface to trigger more efficient CDC or ADCC. The mechanisms of the P₄N-induced Ig class switch and somatic hypermutation involve the Activin A stimulated monocyte release of BAFF via the ALK4/Smad3 pathway (FIG. 5). BAFF up-regulates the expression of the transcription factor Pax5/BSAP, which sequentially increases the transcription of activation-induced cytidine deaminase (AID), an RNA editing enzyme responsible for IgH class switch recombination and somatic hypermutation (40-43). Moreover, class switch recombination must be supported by GLTs splicing, which are involved in recruiting AID to S regions. Activin A also has been reported to induce the class switching of Ig to IgA and IgG2b in B cells through Smad2/3 signaling by up-regulating the expression of Ig germline transcript α (GLTα) and post-switch transcript α (PSTα) (44). By these mechanisms, P₄N could promote B cell proliferation, differentiation and function to produce higher titer and higher affinity autoantibodies against tumor growth.

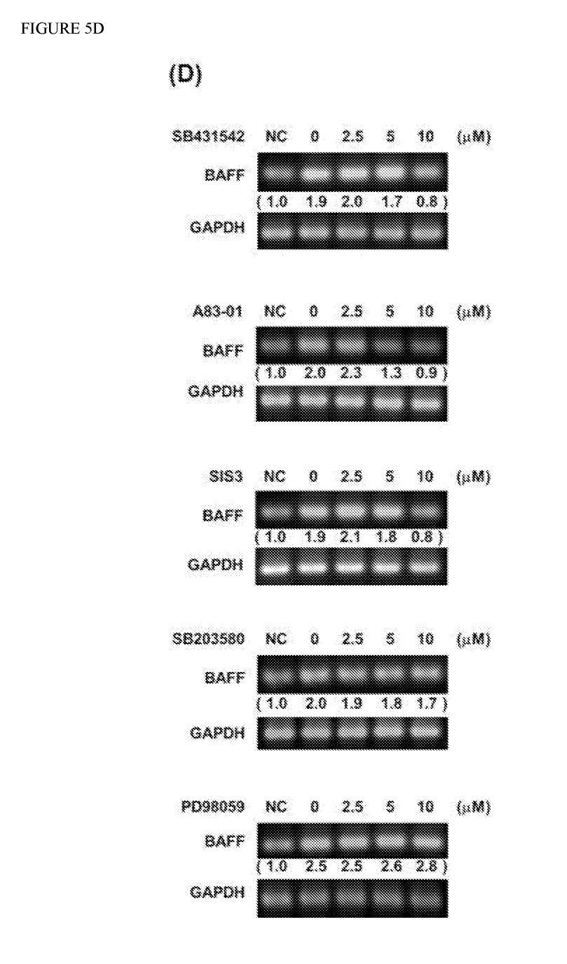

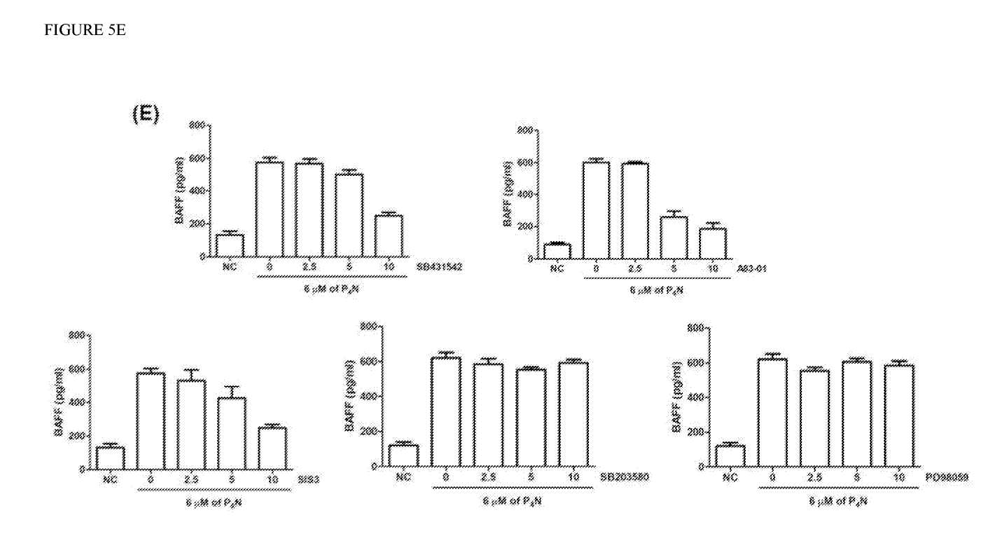

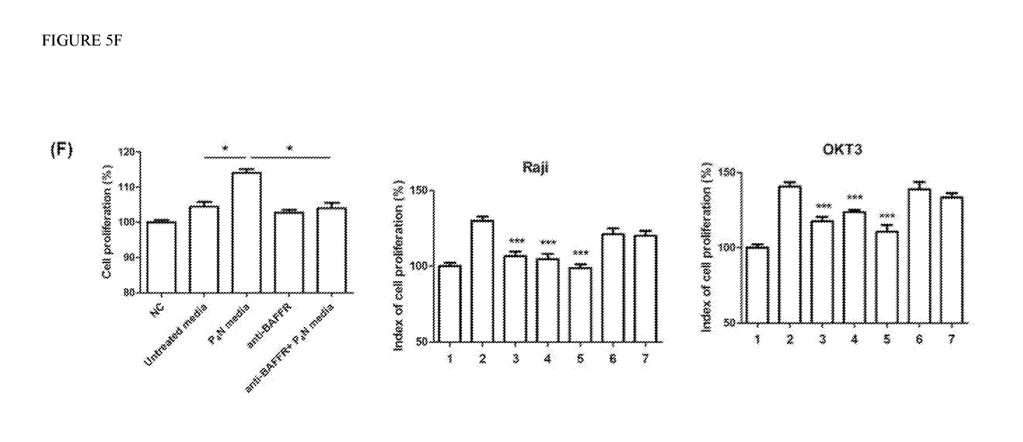

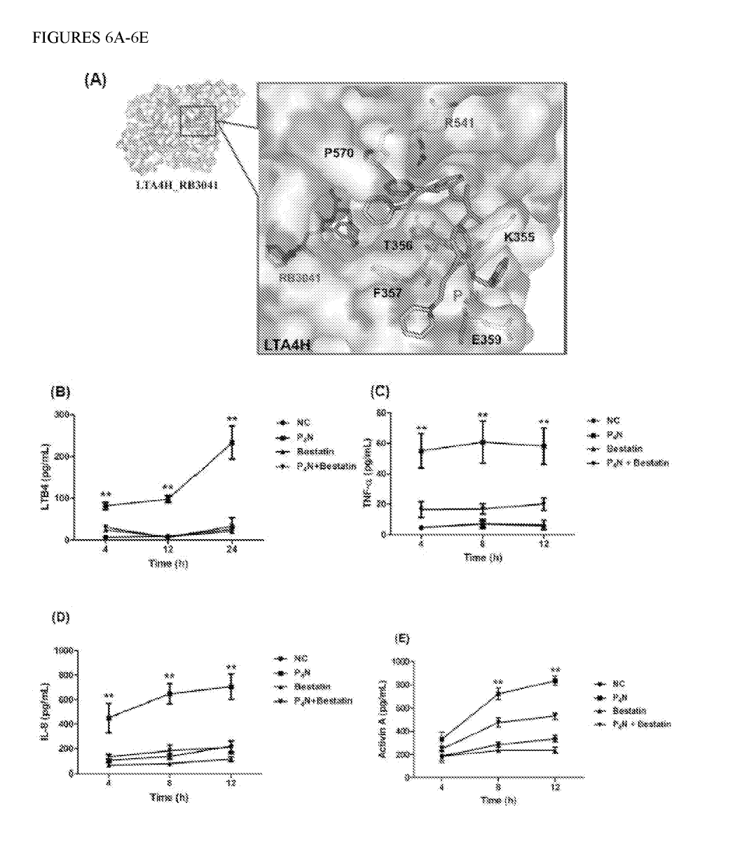

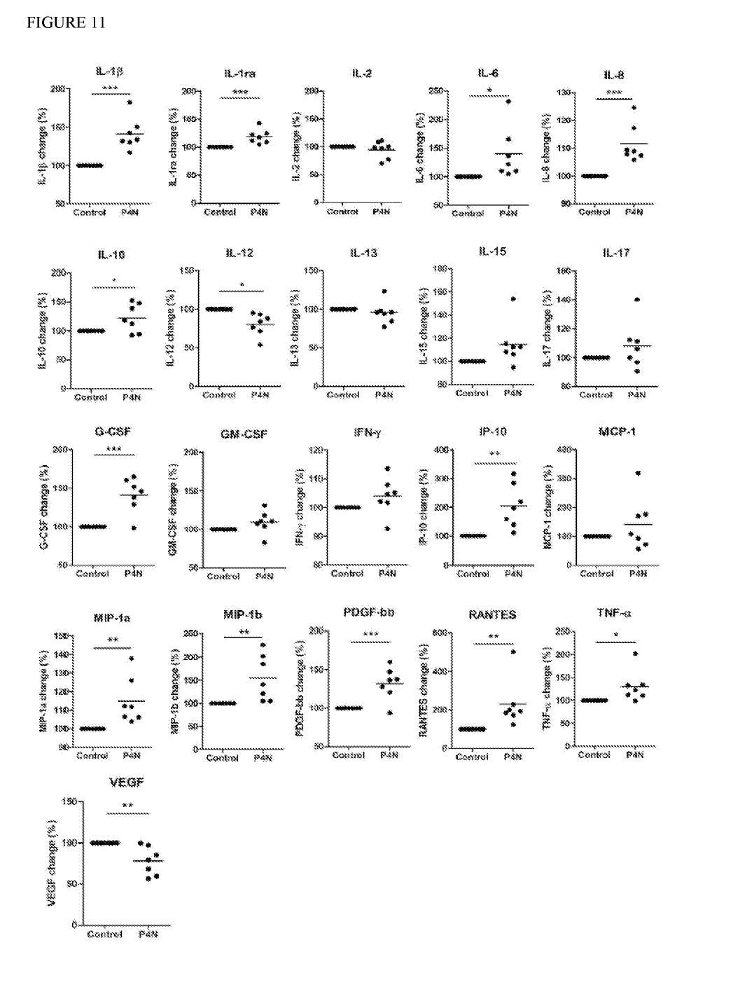

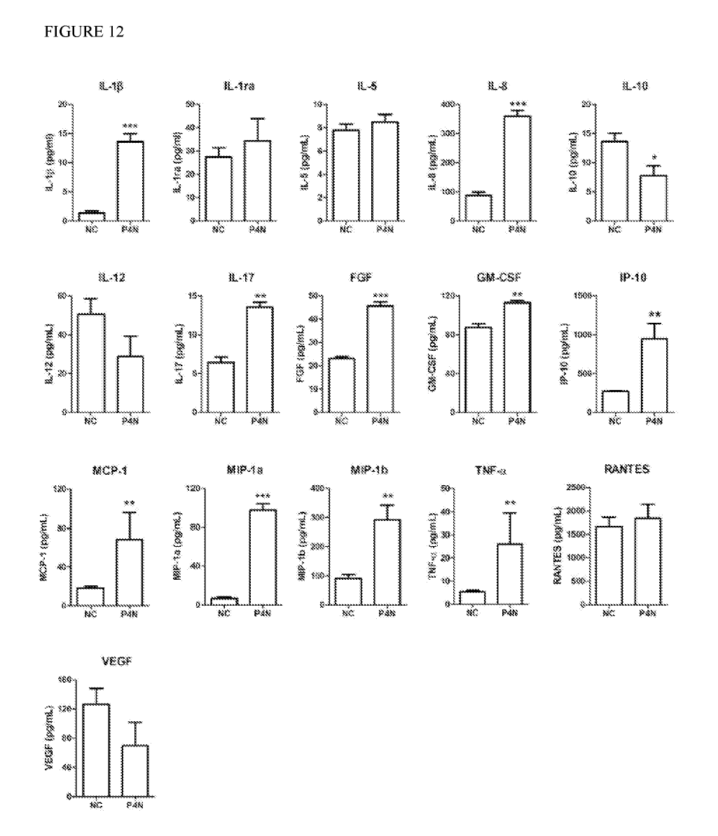

In accordance with the inventive methods, P₄N directly interacted with and activated LTA4H to produce LTB4 (FIG. 6) and induce monocytes to release pro-inflammatory cytokines and chemokines (FIG. 12). Subsequently, Activin A stimulated monocytes in an autocrine manner to release BAFF via the ALK4/Smad3 pathway and activated B cells (FIG. 5). According to the docking results, this unique function of P₄N might be a result of its structure (FIG. 6A). Unlike NDGA, which inhibits LTB4 production (32), the NDGA derivative P₄N binds differently to LTA4H and acts as an activator and immune mediator to produce more LTB4 (FIG. 6B). LTB4 is transported out of the cells and binds to leukotriene B4 receptors expressed primarily in leukocytes and mediates the monocytic up-regulation of TNF-α, IL-1β and IL-6 (45-47). As in the published literature, the inflammatory mediators such as TNF-α, IL-1β, interferon-γ (IFN-γ) and lipopolysaccharide (LPS), markedly enhance the production of Activin A (48, 49). Here, our results clearly indicate that an LTA4H activator can act on monocytes to stimulate the production of LTB4, resulting in B cell proliferation and an increase of Ig production by serial production of autocrine signal mediators such as TNF-α, Activin A, and paracrine BAFF.

### EXAMPLES

Reagents. P₄N was synthesized from NDGA by the Hwu lab and provided for this study by RCCH. SB431542 (ALK4 inhibitor), SIS-3 (Smad3 inhibitor), SB203580 (p38 inhibitor) and PD98059 (MAPK/ERK kinase inhibitor) were purchased from Sigma Aldrich. A83-01 (ALK4 inhibitor) was procured from R&D Systems.

CT-26 and THP-1 cells were purchased from Bioresource Collection and Research Center (BCRC, Hsinchu, Taiwan). CT-26 and THP-1 cells were maintained in the culture media according to the BCRC culture protocols. In general, they were maintained in a culture medium (RPMI-1640 (Invitrogen, Carlsbad, Calif.) supplemented with 4.5 g/L glucose (MP Biomedicals, Santa Ana, Ohio), 10 mM HEPES (MP Biomedicals), 1.0 mM sodium pyruvate (MP Biomedicals), 0.05 mM 2-mercaptoethanol (AMRESCO, Ohio, USA), 10% fetal calf serum (Invitrogen), and 1% penicillin-streptomycin (Invitrogen)).

PBMCs were isolated from the blood of healthy human donors by density separation over Lymphoprep™ (Axis-Shield, Oslo, Norway). Mononuclear cells at the interface were carefully transferred into a centrifuge tube, and then treated with ACK hemolysis buffer (0.15 M NH₄Cl, 10 mM KHCO₃, 0.1 mM Na₂EDTA in distilled water) and washed twice with PBS buffer and cultured in growth medium.

Total B-cells were isolated from human PBMCs or mouse splenocytes by negative selection using a magnetic sorting device (Miltenyi Biotec, Auburn, Calif.). Briefly, PBMCs were incubated with a cocktail of biotin-conjugated antibodies, followed by microbead-conjugated anti-biotin Abs for magnetic depletion. B-cells were eluted according to the manufacturer's protocols. Human blood samples were collected from healthy individuals following an IRB-approved protocol and in agreement with the Committee for Research Ethics Board of Chung Shan Medical University Hospital.

Animals. Female BALB/c and BALB/c/nu mice were purchased from The National Laboratory Animal Center (Taipei, Taiwan) and maintained on a 12:12-h light:dark cycle in an animal environmental control chamber (Micro-VENT IVC Systems, Allentown, N.J.). Humane animal care was ensured by use of the institutional guidelines of National Chiao Tung University (NCTU). The Committee on Animal Experimentation of the Center for Experimental Animals of NCTU approved all studies and procedures.

P₄N treatments in vivo. BALB/c or BALB/c/nu mice (6 week old) were inoculated subcutaneously with 1×10⁶ CT-26 cells in 100 μl PBS. When the average tumor mass reached 50 mm³, the animals were treated with 2.5 mg/kg or 5 mg/kg of P₄N in one intratumoral injection. Subsequently, the tumor volumes were measured using a caliper every 2 days, and the volumes were calculated using the following formula: volume (mm³)=length×width×height. Before sacrifice, the sera of the mice were collected for experimental use.

In vivo cell depletion. CD8+ T and B cells were depleted by monoclonal antibodies following Dr. Carmi Y's protocol (57). CD8+ T cells of was depleted by intraperitoneal (i.p.) injection of 500 μg per mouse anti-CD8 (YST-169.4) monoclonal antibodies (BioXcell, West Lebanon, N.H.) 3 days before tumor inoculation and every 3 days thereafter. For B cell depletion, 300 μg per mouse anti-CD19 (1D3) and 300 μg per mouse anti-220 (RA3.3A1/6.1) monoclonal antibodies (BioXcell) were i.p. injected 3 weeks before tumor inoculation and every 5 days thereafter.

Sera transfer in vivo. In a metastatic model, BALB/c mice were intravenously injected with 1×10⁶ CT-26 cells in 100 μl PBS via the tail vein, and then treated with 100 μl anti-sera once every week. Upon death of the mice, the lungs were dissected, observed and photographed. Three mice in the P₄N anti-sera group were sacrificed to 24 day after the injection. Tumor nodules on the lungs were counted, and lung weights were determined. Survival of the mice was noted, and rates of survival were calculated.

In a subcutaneous tumor model, the nude mice bearing ˜50 mm³ CT-26 tumors were injected intravenously with 100 μl of the different anti-sera once every week. The tumor volumes were measured as described above.

Immunohistochemical staining. Tumors isolated from P₄N and vehicle-treated mice were embedded in paraffin and thin sections were made and processed for immunohistochemical staining. The sections were probed with rat monoclonal anti-F4/80 (1:50 dilution; GeneTex, Irvine, Calif.) and anti-CD20 (1:200 dilution; Santa Cruz Biotechnology, Dallas, Tex.), hamster monoclonal anti-CD11c (1:25 dilution; GeneTex), rabbit monoclonal anti-CD3 (1:50 dilution; GeneTex), rabbit polyclonal anti-TNF-α and anti-IL-8 antibody (1:50 dilution; Assay BioTech. Sunnyvale, Calif.) at 4° C. overnight, and the detection antibodies were recognized using a horseradish peroxidase (HRP)-conjugated anti-rat, anti-hamster, or anti-rabbit IgG antibody (1/1,500 dilution; Santa Cruz Biotechnology). The immune complexes in the sections were visualized using the LSAB2 system (DAKO, Carpinteria, Calif.). The sections were counterstained with hematoxylin, mounted, observed under a light microscope at a magnification of 400×, and photographed.

Sections of CT-26 tumors from untreated mice were incubated with a 200-fold dilution of normal sera, PBS anti-sera or P₄N anti-sera at 4° C. overnight. Sequentially, the sections were recognized using an HRP-conjugated anti-mouse IgG antibody (1:1500 dilution; Santa Cruz Biotechnology), and then developed, counterstained and photographed as described above.

Titers and Ig classes of antitumor autoantibodies. Blood was collected weekly from CT-26-bearing mice treated with PBS or 5 mg/kg P₄N, and the titers of CT-26-specific antibodies in the sera were measured by the following method. The wells of 96-well culture plates were seeded with CT-26 (10⁶ cells/well) or mouse mammary gland adenocarcinoma JC cells (10⁶ cells/well). On the following day, the cells were fixed with 4% paraformaldehyde, washed, and blocked with 300 μl of 2% skim milk PBST (PBS buffer with 0.05% tween-20) for 1 h. One hundred microliters of 6400-fold diluted sera in PBS containing 0.5% skim milk were loaded into each well and incubated at room temperature for 2 h. After washing 3 times, 100 μl HRP-conjugated anti-mouse Ig antibody (1:10000 dilution; Sigma-Aldrich, St. Louis, Mo.) were added to each well and incubated for 1 h. After washing 3 times, 100 μL of NeA-Blue (Clinical Science Products, Inc. Mansfield, Mass.) was added to each well, incubated for 20 minutes and stopped using 100 μL 1 N HCl. The optical density was measured at 450 nm using an ELISA reader (Tecan, Mannedorf, Switzerland). The isotypes of specific anti-CT-26 cell antibodies in anti-sera (1:1600 dilution) were determined by using HRP-conjugated specific anti-mouse IgM, IgG1, IgG2a, IgG2b or IgA antibodies (Acris, Herford, Germany).

Immunofluorescence analysis of cell surface tumor antigens. CT-26 cells were seeded on glass cover slides, and fixed with 4% (w/w) paraformaldehyde The fixed cells were then incubated with normal sera, PBS anti-sera or P₄N anti-sera for 1 h and detected with secondary antibodies conjugated to Alexa Fluor 568 (Molecular Probes, Eugene, USA). Alexa Fluor 488-conjugated Concanavalin A (ConA; Molecular Probes) and DAPI (Molecular Probes) were used to label the plasma membrane and nucleus. The images of tumor antigens recognized by the anti-sera were photographed at a 400× magnification using a Zeiss LSM 510 META confocal microscope (Carl Zeiss, Jena, Germany).

Western blot analysis. The membrane proteins of 3×107 CT-26 cells were extracted and harvested with the Mem-PER Eukaryotic Membrane Protein Extraction Reagent Kit (Thermo Fisher Scientific, Rockford, Ill.), following the manufacturer's instructions. Fifty micrograms of total membrane protein was subjected to SDS-PAGE, transferred to Nitrocellulose Blotting Membrane (GE, Pittsburgh, Pa.) and probed with normal sera, PBS anti-sera or P₄N anti-sera. The membranes were then blocked with 2% silk milk in PBST, incubated with HRP-conjugated anti-mouse Ig antibody (1:10000 dilution; Sigma-Aldrich), and developed with the WesternBright™ ECL Western blotting detection kit (Advansta, Menlo Park, Calif.). Antibody bound proteins were visualized by the Hansor Luminescence Image system (Hansor, Taichung, Taiwan). E-cadherin proteins in all samples were probed by rabbit polyclonal anti-E-cadherin antibody (1:1000 dilution; GeneTex) and HRP-conjugated anti-rabbit Ig antibody (1:10000 dilution; Sigma-Aldrich).

Co-immunoprecipitation assay. Five microliters of normal sera, PBS anti-sera or P₄N anti-sera were mixed with 100 μl of the protein G agarose (Merck Millipore, Darmstadt, Germany), and then covalently linked with disuccinimidyl suberate (DSS, Thermo Fisher Scientific). After washing, 5 μl of protein G agarose conjugated sera were incubated with 400 μl membrane proteins at room temperature. The immune complexes were then washed three times with lysis buffer and eluted with elution buffer (Thermo Fisher Scientific). The precipitated samples were then heated in reducing sample buffer and resolved by the SDS-PAGE.

Antigen identification by UPLC/HRMS/MS. Proteins were excised from SDS polyacrylamide gels, digested with the In-Gel Tryptic Digestion Kit (Thermo Fisher Scientific), and identified by UPLC/HRMS/MS (Bruker BioSpin, Rheinstetten, Germany). Peptide sequence information was used to search sequences in the protein database of NCBI using BLAST (blast.ncbi.nlm.nih.gov/Blast.cgi).

Gene expression profiling and analysis. Amplification and labeling of RNA was performed with the Illumina®TotalPrep RNA Amplification Kit from Life Technologies (Ambion, Applied Biosystems, Foster City, Calif.) using 150 ng of RNA per sample. Labeled RNA (750 ng) was hybridized to Illumina HT-12 v4 Expression BeadChips (˜48,000 probes) and processed according to the manufacturer's protocol. Expression data underwent quality control and normalization by GenomeStudio (Illumina, San Diego, Calif.). Genes differentially expressed in the cells treated with P₄N relative to the untreated cells were identified, with special emphasis given to genes involved in cytokine-cytokine receptor interaction (KEGG pathway). Genes with a p-value of p<0.05 and fold change ≥0.4 as up-regulated were considered to be differentially expressed genes. The identified genes were subjected to the Database for Annotation, Visualization and Integrated Discovery (DAVID) (david.abcc.ncifcrf.gov/) for Gene Ontology (GO) and KEGG pathway enrichment analysis. A P-value <0.05 was set as threshold of enrichment analysis.

Reverse transcription PCR. Human PBMCs or THP-1 cells were treated with P₄N, and then the mRNA expression of INHBA and BAFF in these cells was measured by reverse transcription PCR. Briefly, total cellular RNA was extracted with Trizol reagent (Invitrogen) and reverse-transcribed into cDNA using the Superscript RT-kit (Invitrogen). The cDNA of INHBA and BAFF were then amplified by PCR. GAPDH cDNA in the samples was used to normalize the loading amounts in each reaction. The primer sets used in this study are shown in Table 1. Finally, PCR products were resolved by electrophoresis on 2% agarose gels and visualized with ethidium bromide.

TABLE 1
Primers used in PCR Analysis

| | | | GenBank |
|---|---|---|---|
| | | | accession |
| Primers | Forward | Reverse | number |
| hINHBA | 5′- | 5′- | NM_002192.2 |
| | GCCGAGTCAGGAACAGCCAG- | TTTCTTCTTCTTCTTGCCCAGGA- | |
| | 3′ (SEQ ID NO: 1) | 3′ (SEQ ID NO: 2) | |
| hBAFF | 5′- | 5′-TGGTAGAAAGACACCACCG-3′ | AF116456.1 |
| | ATGGATGACTCCACAGAAAGG- | (SEQ ID NO: 4) | |
| | 3′ (SEQ ID NO: 3) | | |
| mINHBA | 5′-ATGCCCTTGCTTTGGCTGAG- | 5′- | NM_008380.1 |
| | 3′ (SEQ ID NO: 5) | TTCAAGTGCAGCATGTTTAAGAT- | |
| | | 3′ (SEQ ID NO: 6) | |
| mBAFF | 5′- | 5′-GGACATCGCTGTGAAACTGC- | AF119383.1 |
| | ATGGATGAGTCTGCAAAGACC- | 3′ (SEQ ID NO: 8) | |
| | 3′ (SEQ ID NO: 7) | | |

Cell proliferation assay. Raji or OKT3 cells (1×10⁴/well) were seeded and treated with THP-1-conditioned media. After 48 h the number of viable cells was determined by MTT assay. The indices of cell proliferation were calculated as previously described.

ELISA assays. THP-1 cells (1×10⁶ cells/ml/well) in a 24-well culture plate were pretreated with 10 μM bestatin (a LTA4H inhibitor, Sigma-Aldrich) for 2 h and then treated with 3 μM P₄N for various time intervals. The levels of LTB4 in the culture media were determined by the LTB4 (Enzo Life Sciences, Farmingdale, N.Y.) (R&D) ELISA Kits. The amount of TNF-α, IL-8 and Activin A in cultured media were measured in a similar manner.

Statistical analyses. The results are presented as the mean±SEM. The statistical significance was evaluated using Student's t-test; p<0.05 was considered significant.

PBMC proliferation assay. Two hundred microliters of PBMCs (5×10⁴ cells/ml) were seeded in each well of a 96-well culture plate and treated with increasing concentrations of P₄N (0.38, 0.75, 1.5, 3 or 6 μM) for 48 h. The number of viable cells was evaluated by MTT assay and a cell proliferation index was calculated as follows: Proliferation index=(OD₅₉₅ of sample)/(OD₅₉₅ of the untreated cells)×100%. PBMCs were also treated with or without P₄N (0.75, 1.5 or 3 μM) for 48 h, and probed with anti-CD20 antibodies (Roche, Mannheim, Germany) and FITC-conjugated goat anti-human IgG polyclonal antibodies (SouthernBiotech, Birmingham, Ala.). The percentage of B cells in treated PBMCs was determined by flow cytometry (BD Biosciences, Mountain View, Calif.), and the indices of B cell proliferation were calculated as follows: (the number of PBMCs×the percentage of B cells in treated PBMCs)/(the number of PBMCs×the percentage of B cells in untreated PBMCs).

The effect of P₄N on changes in B cell lineage in vitro. Single cell suspensions of mouse splenocytes were obtained by grinding the spleens from healthy mice. The suspended splenocytes were treated with or without 0.1 or 0.2 μM of P₄N for 48 h, and the percentage of naïve B cells, activated B cells, B1 cells or Breg cells were measured after staining with the following fluorochrome conjugates using flow cytometry (BD Biosciences): anti-CD19-FITC, anti-CD-23-APC, anti-CD-38-APC, anti-CD5-PE or anti-CD1d-Alexa 647 (BioLegend, San Diego, Calif.). The cell types were defined as follows: naïve B cells (CD19+CD23+), activated B cells (CD19+CD38+), B1 cells (CD19+CD5+) and Breg cells (CD19+CD5+CD1d+). The indices of different types of B cell proliferation were calculated as follows: (the number of splenocytes×the percentage of each B cell population in treated splenocytes)/(the number of splenocytes×the percentage of each B cell population in untreated splenocytes).

In vivo effects of P₄N on antibody production and B cell differentiation. Mice were immunized and boosted with 100 μg EGFP in 100 μl of PBS by intraperitoneal injection once every week and simultaneously treated with or without 5 mg/kg P₄N once or three times per week. Blood samples were collected from the retro-orbital plexus of the mice every week. The titers of anti-EGFP antibodies in sera were measured by enzyme-linked immunosorbent assay (ELISA). Briefly, each well of a microtiter plate (Nunc, Wiesbaden, Germany) was coated with 100 ng of rEGFP in 100 μl of PBS. The sera were 6400× diluted by PBS and added to the antigen-coated wells, and their titers were measured as described above. The isotypes of anti-EGFP antibodies in sera were also measured as described previously.

The splenocytes of P₄N-treated or untreated mice were harvested and probed with anti-CD19-FITC, anti-CD23-APC, anti-CD38-APC, anti-CD27-APC, anti-CD138-APC, anti-CD5-PE or anti-CD1d-Alexa 647 (BioLegend) antibodies and analyzed by flow cytometry (BD Biosciences). The definitions of the cell types were as described above, except for memory B cells (CD19+CD27+) and plasma cells (CD19+CD138+). The indices of different types of B cell proliferation were calculated as follows: (the number of splenocytes×the percentage of each B cell population in treated splenocytes)/(the number of splenocytes×the percentage of each B cell population in untreated splenocytes).

The effects of P₄N on the profile of cytokine expression. PBMCs (2×10⁶ cells/ml) from seven healthy individuals were seeded in each well of a 24-well microplate and treated with 3 μM of P₄N for 24 h. Cell supernatants were collected for analysis of cytokine levels by a cytokine multiplex assay using the Bio-Plex Pro Human Cytokine Standard Group I 27-Plex, following the manufacturer's protocol (Bio-Rad, Hercules, Calif.). The change levels of cytokine were calculated as follows: Change level=(value of the P₄N-treated cells)/(value of the untreated cells)×100%.

### Example 1

P₄N activated humoral immune response leading to suppression of tumor growth in vivo.

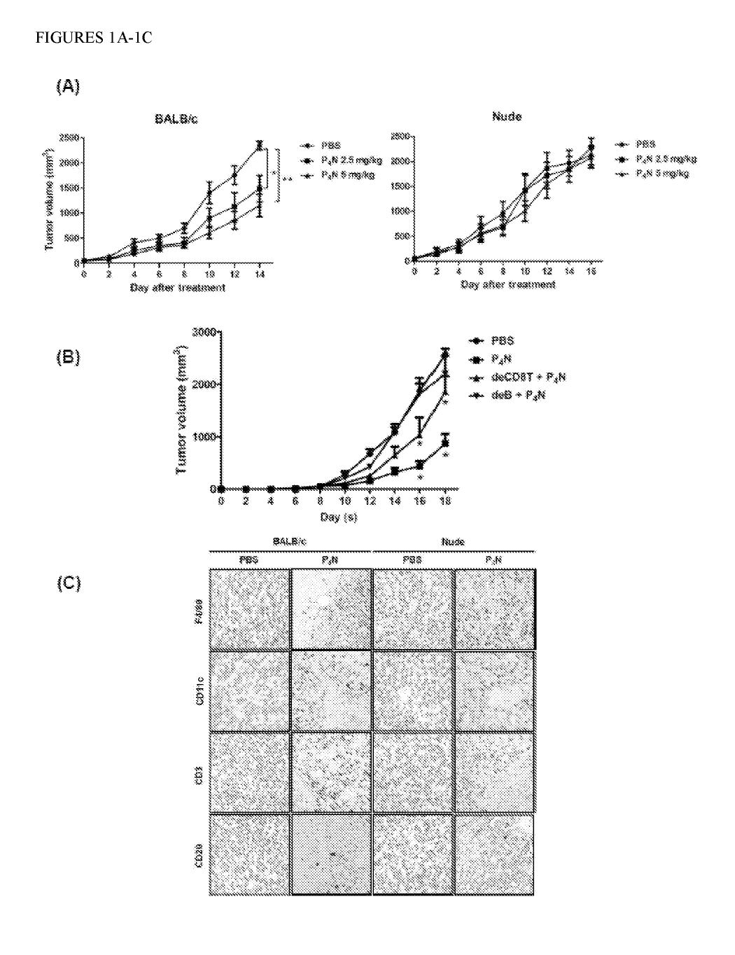

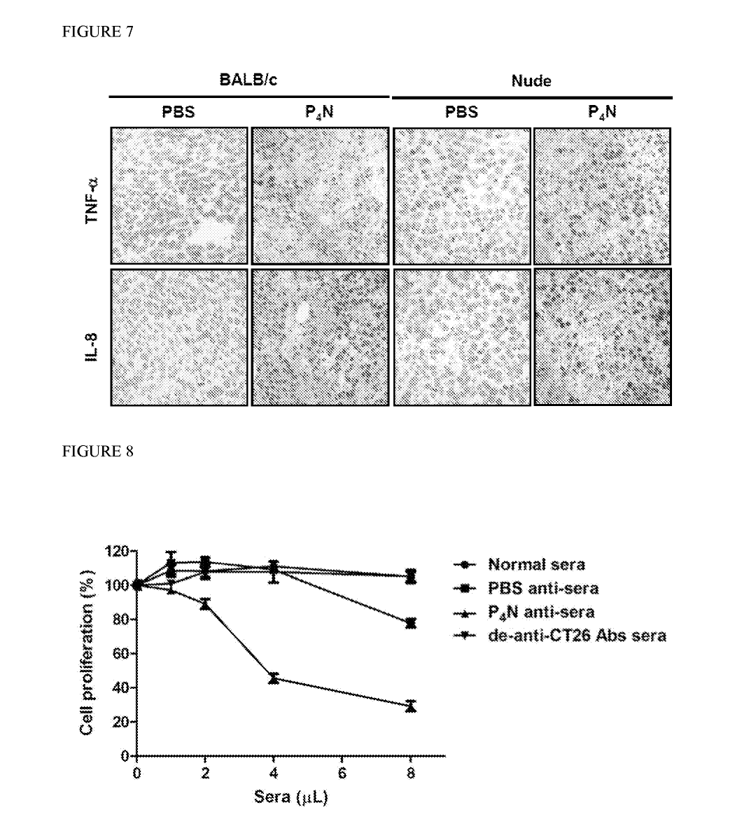

To examine whether low-dose P₄N causes immunoregulatory activity for the suppression of tumor growth, the effects of one intratumoral injection with 2.5 mg/kg or 5 mg/kg of P₄N in CT-26 tumors in BALB/c or nude mice were monitored. Both doses of P₄N significantly inhibited the growth of CT26 tumors in BALB/c mice, but had no effect on the growth of CT26 tumors in the immunodeficient nude mice (FIG. 1A). As shown in FIG. 1B, CD8+ T cell-depletion only attenuated the effect of P₄N-induced tumor inhibition. In contrast, B cell-depletion abolished the effect of P₄N-induced tumor inhibition (FIG. 1B). In addition, B cell-depletion caused no significant difference between the mice with P₄N or PBS treatments in the tumor growths that indicated the tumor suppressive effects of P₄N intratumoral injections did not result from the cytotoxicity of this drug. Therefore, these results revealed that a humoral immune response may play a major role in P₄N-induced tumor inhibition. Using an IHC assay, it was shown that P₄N treatments led to the infiltration of immune cells, such as macrophages, DC, T or B cells, into the tumor area (FIG. 1C). There was no significant difference between the two mouse strains in terms of the levels of macrophages and DCs in the tumors, but the levels of T and B cells in the tumors were higher in BALB/c mice than nude mice. In addition, it was found that P₄N treatments enhanced the expression levels of TNF-α and IL-8 in both strains of mice (FIG. 7). Taken together, these results indicate that low dose P₄N elicits certain immune responses in tumors, but only suppresses tumor growth in BALB/c mice, where adaptive immunity may play an important role in the low dose P₄N-induced tumor suppression.

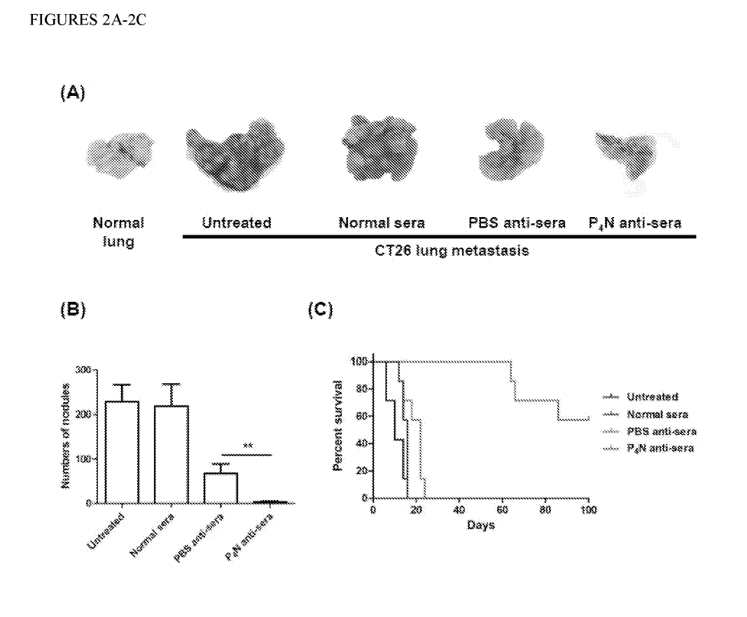

To verify whether humoral immunity is involved in the antitumor activity of low dose P₄N, sera isolated from P₄N or vehicle (PBS) treated CT-26 tumor-bearing mice were passively transferred into BALB/c mice with inoculations of metastatic CT-26 cells. On day 18 after tumor injection, CT-26 cells in untreated mice developed serious lung metastasis, and metastatic nodules surrounded the lungs (FIG. 2A). Administration with anti-sera derived from PBS treated mice significantly decreased the colonization of CT-26 cells from circulation to the lung. P₄N derived anti-sera displayed an even stronger effect, dramatically inhibiting tumor colonization and abolishing the colonization of metastatic cells. The CT-26 cell metastatic nodules in the lungs of each group were counted and the lungs were weighed. Analysis of the results confirmed that P₄N anti-sera treatments reduced tumor colonization (FIG. 2B). All mice treated with P₄N anti-sera survived more than 60 days, a survival time 3 times that of the control groups (FIG. 2C). These results suggested that the inhibition of tumor colonization by P₄N anti-sera might be the result of P₄N-induced EAAs in the sera.

### Example 2

The effects of P₄N on production and activity of anti-tumor autoantibodies.

To eliminate the influence of T cells, the anti-sera were injected into CT-26 tumor-containing immunodeficient mice. P₄N anti-sera still significantly suppressed tumor growth in these mice, while PBS anti-sera had no significant effects on tumor growth (FIG. 3A). Characterize of the anti-sera revealed that the titers of specific anti-CT-26 antibodies in P₄N anti-sera were higher than in PBS anti-sera, regardless of the time of harvest (FIG. 3B) and the major classes of increased antibodies in P₄N anti-sera were IgG1 and IgA (FIG. 3C). Like normal mouse sera, PBS anti-sera only somewhat recognized the tumor cells in CT-26 tumor tissue, however, the P₄N anti-sera displayed a strong binding affinity to the tumor cells in CT-26 tumor tissue (FIG. 3D). In addition, P₄N anti-sera also provide more efficient cytotoxicity (FIG. 8).

Of interest are the antigens that are recognized by EAAs in sera derived from tumor-bearing mice. FIG. 3E shows that although both anti-sera recognized surface antigens on CT-26 cells, P₄N anti-sera had a better ability than PBS anti-sera. The autoantibody-bound antigens on the plasma membrane were displayed in a spot-shape that could imply that they may associate with other proteins to form complexes on the cell surface (FIG. 3F). A western blotting assay determined that the autoantibodies in the anti-sera recognized 78 kDa and 55 kDa proteins on the membranes of CT-26 cells (FIG. 3G). The antigen profile on the cell membrane was obtained by co-immunoprecipitation (IP) of a membrane protein extract with the anti-sera. The results showed that more than 6 proteins were co-precipitated with the anti-sera targeting proteins (FIG. 3H), which indicating that the 78 kDa and 55 kDa proteins recognized by the anti-sera are associated with other proteins, forming a complex on the cell surface. The 78 kDa and 55 kDa proteins were subsequently identified by UPLC/HRMS/MS as GRP78 and FIFO ATP synthase, respectively (FIG. 3I). These results indicated that P₄N treatments enhance the production and activity of anti-GRP78 and anti-FIFO ATP synthase autoantibodies that promote CT-26 tumor inhibition.

### Example 3

The effects of low dose P₄N on B cell proliferation and immunoglobulin regulation.

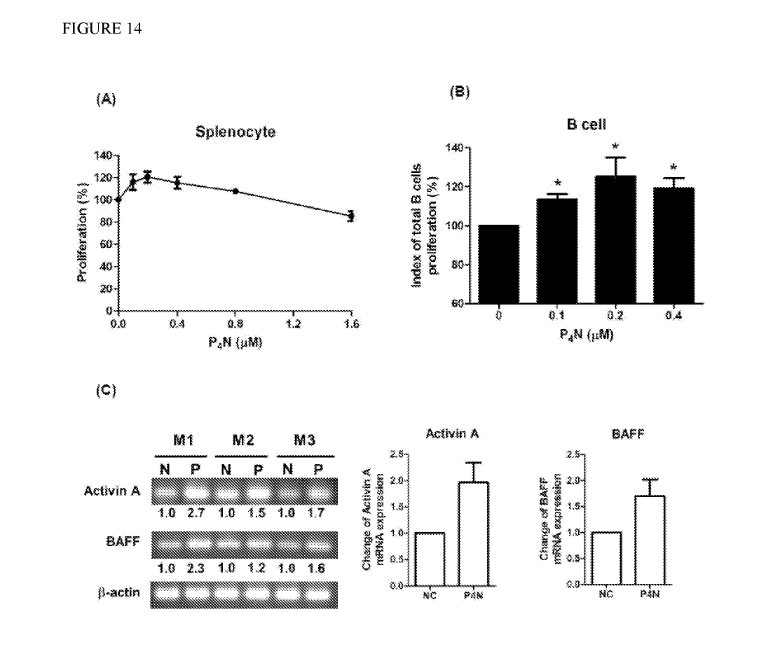

To examine the effects of low dose P₄N on immune modulation, it was determined whether P₄N affected the proliferation of human PBMCs in vitro. As shown in FIG. 9A, PBMC proliferation was increased by 10-35%, depending on the dose of P₄N. After further analysis, it was found that P₄N significantly increased the proliferation of the B cell population in human PBMCs (FIG. 9B). B cell proliferation was also analyzed in mouse splenocytes and the results showed that P₄N increased total B cell proliferation (FIGS. 9C, 14A and 14B). These results indicated that P₄N treatments have similar effects mice and humans. Additionally, the B cell types were determined, and it was found that P₄N increased total B cell proliferation as a result of naïve B cell proliferation and had no effect on the number of activated B cells in the absence of antigen stimulation.

EGFP was used as a model antigen to immunize the mouse to monitor the specific antibody production and B cell differentiation after antigen stimulation. BALB/c mice were immunized and boosted using i.p. injections with the EGFP antigen combined with one or three times weekly P₄N injections. As in previous results, P₄N dramatically enhanced the total specific anti-EGFP Ig production, especially when EGFP was combined with P₄N injections three times a week (FIG. 9D). In addition, immunization with P₄N significantly increased IgG1, IgG2b and IgA production (FIG. 9E). These results indicate that P₄N promotes specific antibody production by up-regulating the Th2-mediated immune response. Moreover, the lineages of the B cell were determined after EGFP immunization, and it was found that both memory and plasma cells were increased (FIG. 9F). Compared to EGFP immunization, P₄N greatly stimulated B cell differentiation to plasma cells, demonstrating that it not only stimulates B cell proliferation but also enhances the process of B cell differentiation. Thus, P₄N treatment could lead animals to produce more antibodies.

### Example 4

Role of monocytes and cytokine expression in P₄N-induced B cell proliferation.

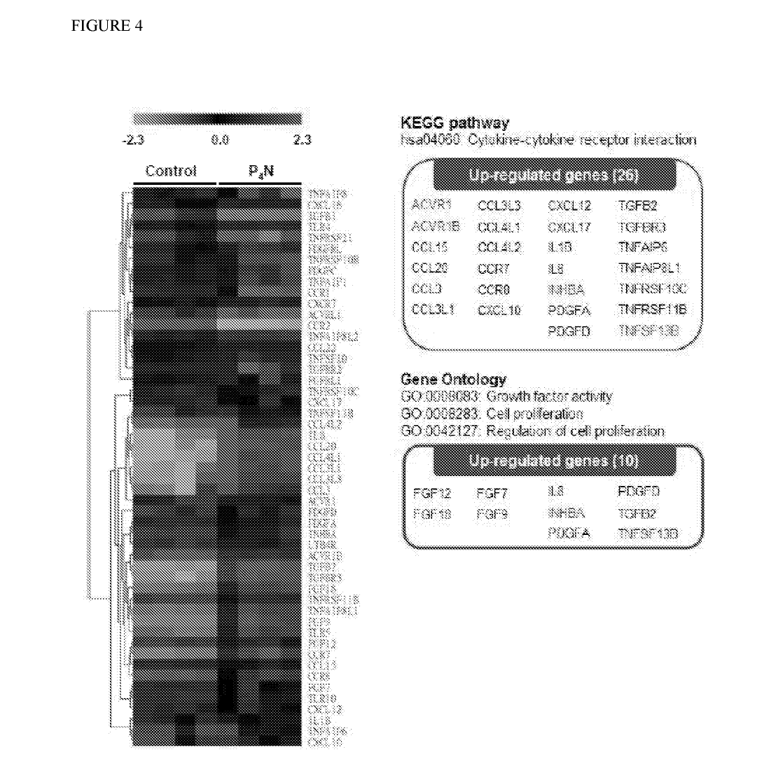

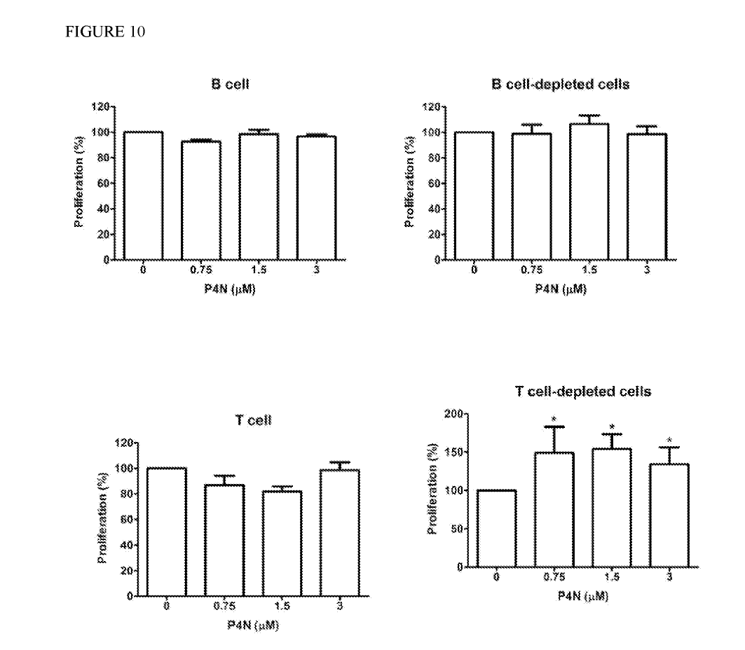

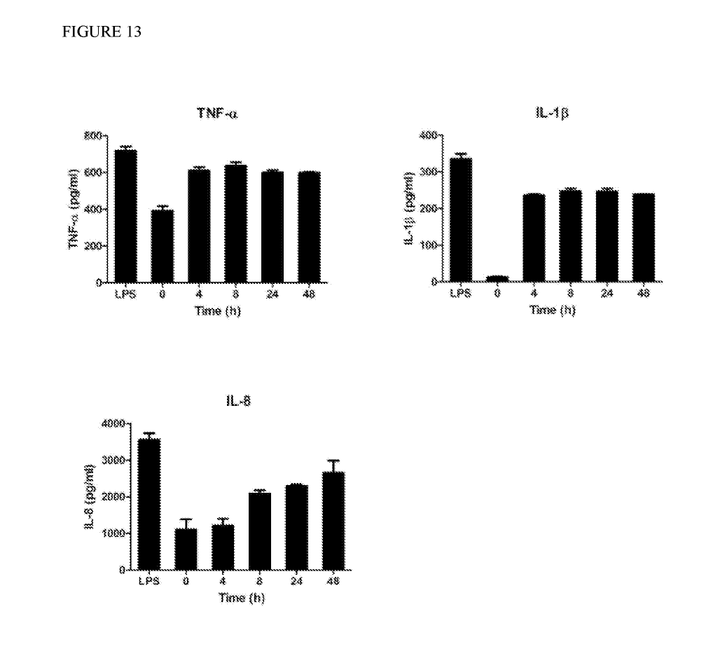

P₄N treatments did not induce the proliferation of isolated T cells or B cells, yet increased the proliferation of T-cell-depleted PBMCs (FIG. 10), indicating that P₄N-induced B cell proliferation may require the presence of monocytes. Examination of the cytokine profiles of PBMCs after P₄N treatment showed that, the levels of pro-inflammatory cytokines (TNF-α, IL-1P and IL-6) and chemokines (IL-8, IP-10, MIP-1α, MIP-1β and RANTES) were significantly increased after P₄N treatment (FIG. 11). Using a Bio-Plex assay for human cytokines to analyze THP-1 monocyte-like cells it was determined that after P₄N treatment, the THP-1 cells increased the expression of pro-inflammatory cytokines and chemokines, similar to PBMCs (FIG. 12). Moreover, P₄N rapidly induced TNF-α and IL-1β expression in THP-1 monocytes within 4 h, and then stimulated IL-8 release after 8 h (FIG. 13), suggesting that P₄N's effect on monocytes results in a cascade of cytokines or chemokines. With no published reports to suggest what factor(s) are involved in P₄N-induced B cell proliferation, the gene expression pattern of monocytes treated with P₄N was analyzed by cDNA microarray to determine which genes are modulated in this process. The results of this analysis revealed 26 up-regulated genes of cytokine-cytokine interaction (KEGG pathway) and the 10 up-regulated genes of growth factor activity (GO: 0008083) in THP-1 cells following P₄N treatment (FIG. 4). Based on these results, the factor most capable of activating B cells without antigen stimulation, BAFF (TNFSF13B) and was chosen for further study along with Activin A (INHBA), because it is associated with BAFF expression.

### Example 5

Effect of P₄N-induced inflammatory monocytes on Activin A and BAFF expression. It has been reported that inflammatory monocytes markedly enhance the production of Activin A, which can stimulate antigen-presenting cells to express BAFF (31). In accordance with these reports, we observed that low dose P₄N induced human PBMCs or mouse splenocytes to increase their gene expression and protein production of Activin A and BAFF (FIG. 5A and FIG. 14C) in a dose-dependent manner (FIG. 5B). Moreover, P₄N up-regulated mRNA expression of Activin A at 2 h, however BAFF expression was elevated at 8 h (FIG. 5C). This implies that in P₄N-treated monocytes Activin A release stimulates the expression of BAFF.

### Example 6

Involvement of the ALK4/Smad3 pathway in Activin A-induced BAFF expression. To determine the mechanism of Activin A-induced BAFF expression after treatment with P₄N, SB431542 (an ALK4 inhibitor), A83-01 (an ALK4 inhibitor), SIS3 (a Smad3 inhibitor), SB203580 (a p38 inhibitor) and PD98059 (an ERK inhibitor) were used to examine the pathways involved. FIGS. 5D and 5E show that SB431542, A83-01 and SIS3 significantly reduced BAFF transcription and protein expression, whereas SB203580 and PD98059 had fewer effects, suggesting that the ALK4/Smad3 pathway mediates the Activin A-induced expression of BAFF. Furthermore, conditioned medium from THP-1 cells treated with P₄N increased the proliferation of Raji or OKT3 cells, whereas conditioned medium from THP-1 cells treated with P₄N and the inhibitors SB431542, A83-01 or SIS3 lost its activity (FIG. 5F). In this conditioned media, since the concentration of BAFF was reduced by the inhibitors, P₄N-induced B cell proliferation is likely due to the action BAFF that is induced by Activin A through the ALK4/Smad3 pathway.

### Example 7

P₄N activated leukotriene A4 hydrolase and regulated monocyte inflammation.

The inventors wished to determine the molecular targets of P₄N that mediate the response seen in this study. According to docking analysis using Swiss Target Prediction and iGEMDOCK software identified the enzyme leukotriene A4 hydrolase (LTA4H) as a possible target of P₄N. LTA4H catalyzes the final step in the biosynthesis of the pro-inflammatory mediator leukotriene B4. P₄N docked on a non-catalytic site of LTA4H when compared to the small molecule active site inhibitor RB3021 (FIG. 6A). The effect of P₄N on the activity of LTA4H was examined, and FIG. 6B shows that P₄N increased the production of its reaction product LTB4, These results revealed that the NDGA-derivative P₄N is a LTA4H activator and not a LTA4H inhibitor like its maternal molecule NDGA (32, 33). The pro-inflammatory mediator LTB4 induces the expression of cytokines and chemokines, however, it can also be induced by pro-inflammatory cytokines and chemokines. We therefore used the LTA4H inhibitor, Bestatin to distinguish whether P₄N induced the expression of the pro-inflammatory cytokines and chemokines through LTA4H activation, or if it increased the activity of LTA4H through the induction of pro-inflammatory cytokines and chemokines. As shown in FIGS. 6C and D the expression of TNF-α and IL-8 were not suppressed by Bestatin, but increased following P₄N treatment Thus, it appears P₄N first activates LTA4H to increase LTB4 production, and then LTB4 stimulates the expression of these pro-inflammatory cytokines and chemokines, as shown in FIGS. 11 and 12. Finally, the results also showed that Bestatin inhibited the P₄N-induced expression of Activin A (FIG. 6E), implicating P₄N-activated LTA4H in the monocyte stimulation of the Activin A/BAFF pathway.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

1. WHO (2015) GLOBOCAN 2012: Estimated Cancer Incidence, Mortality and Prevalence Worldwide in 2012. International Agency for Research on Cancer (IARC). Available at globocan.iarc.fr/Default.aspx (Accessed at 10 Aug. 2015).
2. Chen H, Werner S, Tao S, Zornig I, & Brenner H (2014) Blood autoantibodies against tumor-associated antigens as biomarkers in early detection of colorectal cancer. Cancer letters 346(2):178-187.
3. Babel I, et al. (2009) Identification of tumor-associated autoantigens for the diagnosis of colorectal cancer in serum using high density protein microarrays. Mol Cell Proteomics 8:2382-2395.
4. Ran Y, et al. (2008) Profiling Tumor-Associated Autoantibodies for the Detection of Colon Cancer. Clin Cancer Res 14:2696-2700.
5. Babel I, et al. (2014) Identification of tumor-associated autoantigens for the diagnosis of colorectal cancer in serum using high density protein microarrays. Exp Ther Med 8:1279-1284.
6. Bei R, Masuelli L, Palumbo C, Modesti M, & Modesti A (2009) A common repertoire of autoantibodies is shared by cancer and autoimmune disease patients: Inflammation in their induction and impact on tumor growth. Cancer letters 281(1):8-23.
7. Zhang Y, Gallastegui N, & Rosenblatt J D (2015) Regulatory B cells in anti-tumor immunity. International immunology.
8. Diaz-Zaragoza M, Hernandez-Avila R, Viedma-Rodriguez R, Arenas-Aranda D, & Ostoa-Saloma P (2015) Natural and adaptive IgM antibodies in the recognition of tumor-associated antigens of breast cancer (Review). Oncology reports 34(3):1106-1114.
9. Rasche L, et al. (2015) GRP78-directed immunotherapy in relapsed or refractory multiple myeloma—results from a phase 1 trial with the monoclonal immunoglobulin M antibody PAT-SM6. Haematologica 100(3):377-384.
10. Rasche L, et al. (2013) The natural human IgM antibody PAT-SM6 induces apoptosis in primary human multiple myeloma cells by targeting heat shock protein GRP78. PLoS One 8(5):e63414.
11. Oflazoglu E & Audoly LP (2010) Evolution of anti-CD20 monoclonal antibody therapeutics in oncology. MAbs 2.
12. Bianchini G & Gianni L (2014) The immune system and response to HER2-targeted treatment in breast cancer. Lancet Oncology 2:e58-e68.
13. Taylor R P & Lindorfer M A (2008) Immunotherapeutic mechanisms of anti-CD20 monoclonal antibodies. Curr Opin Immunol 20:444-449.
14. Selenko N, et al. (2001) CD20 antibody (C2B8)-induced apoptosis of lymphoma cells promotes phagocytosis by dendritic cells and cross-priming of CD8+ cytotoxic T cells. Leukemia 15:1619-1626.
15. He Q, et al. (2011) Low-dose paclitaxel enhances the anti-tumor efficacy of GM-CSF surface-modified whole-tumor-cell vaccine in mouse model of prostate cancer. Cancer Immunol Immunother 60:715-730.
16. Sevko A, et al. (2012) Application of paclitaxel in low non-cytotoxic doses supports vaccination with melanoma antigens in normal mice. J Immunotoxicol 9:275-281.
17. Zhong H, et al. (2007) Low-dose paclitaxel prior to intratumoral dendritic cell vaccine modulates intratumoral cytokine network and lung cancer growth. Clin Cancer Res 13:5455-5462.
18. Heylmann D, et al. (2013) Human CD4+CD25+ regulatory T cells are sensitive to low dose cyclophosphamide: implications for the immune response. PLoS One 8:e83384.
19. Dimeloe S, et al. (2014) Human regulatory T cells lack the cyclophosphamide-extruding transporter ABCB1 and are more susceptible to cyclophosphamide-induced apoptosis. Eur J Immunol. 44:3614-3620.
20. Lutz E R, et al. (2014) Immunotherapy converts nonimmunogenic pancreatic tumors into immunogenic foci of immune regulation. Cancer Immunol Res 2:616-631.
21. Sharabi A & Haran-Ghera N (2011) Immune recovery after cyclophosphamide treatment in multiple myeloma: implication for maintenance immunotherapy. Bone marrow Res 2011:269519.
22. Peng S, et al. (2013) Low-dose cyclophosphamide administered as daily or single dose enhances the antitumor effects of a therapeutic HPV vaccine. Cancer Immunol Immunother 62:171-182.
23. Sharabi A, Laronne-Bar-On A, Meshorer A, & Haran-Ghera N (2010) Chemoimmunotherapy reduces the progression of multiple myeloma in a mouse model. Cancer Prev Res 3:1265-1276.
24. Kaneno R, et al. (2011) Chemotherapeutic agents in low noncytotoxic concentrations increase immunogenicity of human colon cancer cells. Cell Oncol (Dordr) 34:97-106.
25. Shurin G V, Tourkova I L, Kaneno R, & Shurin M R (2009) Chemotherapeutic agents in noncytotoxic concentrations increase antigen presentation by dendritic cells via an IL-12-dependent mechanism. J Immunol 183:137-144.
26. Kaneno R, Shurin G V, Tourkova I L, & Shurin M R (2009) Chemomodulation of human dendritic cell function by antineoplastic agents in low noncytotoxic concentrations. J Transl Med 7:58.
27. Dohm J A, et al. (2005) Influence of ions, hydration, and the transcriptional inhibitor P₄N on the conformations of the Sp1 binding site. J Mol Biol 349(4):731-744.
28. Zhang Y, et al. (2012) mTORC1 is a target of nordihydroguaiaretic acid to prevent breast tumor growth in vitro and in vivo. Breast Cancer Res Treat 136(2):379-388.
29. Dohm J A, et al. (2005) Influence of ions, hydration, and the transcriptional inhibitor P₄N on the conformations of the Sp1 binding site. J Mol Biol 349:731-744.
30. Hwu J R, Hsu M H, & Huang R C (2008) New nordihydroguaiaretic acid derivatives as anti-HIV agents. Bioorg Med Chem Lett 18:1884-1888.
31. Kim R I, Seo G Y, & Kim P H (2011) Activin A Stimulates Mouse APCs to Express BAFF via ALK4-Smad3 Pathway. Immune Netw 11(4):196-202.
32. Conti P, et al. (1993) Human recombinant IL-1 receptor antagonist (IL-1Ra) inhibits leukotriene B4 generation from human monocyte suspensions stimulated by lipopolysaccharide (LPS). Clinical and experimental immunology 91(3):526-531.
33. Maloff B L, Fefer D, Cooke G M, & Ackerman N R (1987) Inhibition of LTB4 binding to human neutrophils by nordihydroguaiaretic acid. Agents and actions 21(3-4):358-360.
34. Ogawa K, Funaba M, & Tsujimoto M (2008) A dual role of Activin A in regulating immunoglobulin production of B cells. Journal of leukocyte biology 83(6):1451-1458.
35. Hashimoto 0, et al. (1998) The role of Activin type I receptors in Activin A-induced growth arrest and apoptosis in mouse B-cell hybridoma cells. Cellular signalling 10(10):743-749.
36. Zipori D & Barda-Saad M (2001) Role of Activin A in negative regulation of normal and tumor B lymphocytes. Journal of leukocyte biology 69(6):867-873.
37. Wang B, et al. (2009) Involvement of ERK, Bcl-2 family and caspase 3 in recombinant human Activin A-induced apoptosis in A549. Toxicology 258(2-3):176-183.
38. Koseki T, et al. (1998) Correlation between Bcl-X expression and B-cell hybridoma apoptosis induced by Activin A. Cellular signalling 10(7):517-521.
39. Craxton A, Draves K E, Gruppi A, & Clark E A (2005) BAFF regulates B cell survival by downregulating the BH3-only family member Bim via the ERK pathway. J Exp Med 202(10):1363-1374.
40. Anderson K S, et al. (2015) Autoantibody signature for the serologic detection of ovarian cancer. J Proteome Res 14(1):578-586.
41. Xu Z, et al. (2007) Regulation of aicda expression and AID activity: relevance to somatic hypermutation and class switch DNA recombination. Critical reviews in immunology 27(4):367-397.
42. Stavnezer J & Schrader C E (2014) IgH chain class switch recombination: mechanism and regulation. J Immunol 193(11):5370-5378.
43. Muramatsu M, et al. (2000) Class switch recombination and hypermutation require activation-induced cytidine deaminase (AID), a potential RNA editing enzyme. Cell 102(5):553-563.
44. Lee H J, Seo G Y, Kim H A, & Kim P H (2008) Activin A stimulates IgA expression in mouse B cells. Biochem Biophys Res Commun 366:574-578.
45. Rola-Pleszczynski M & Lemaire I (1985) Leukotrienes augment interleukin 1 production by human monocytes. J Immunol 135(6):3958-3961.
46. Poubelle P E, Stankova J, Grassi J, & Rola-Pleszczynski M (1991) Leukotriene B4 up-regulates IL-6 rather than IL-1 synthesis in human monocytes. Agents and actions 34(1-2):42-45.
47. Xu S, Lu H, Lin J, Chen Z, & Jiang D (2010) Regulation of TNFalpha and IL1beta in rheumatoid arthritis synovial fibroblasts by leukotriene B4. Rheumatology international 30(9):1183-1189.
48. Yoshino 0, et al. (2011) Activin-A is induced by interleukin-1beta and tumor necrosis factor-alpha and enhances the mRNA expression of interleukin-6 and protease-activated receptor-2 and proliferation of stromal cells from endometrioma. Fertility and sterility 96(1):118-121.
49. Jones K L, et al. (2007) Activin A is a critical component of the inflammatory response, and its binding protein, follistatin, reduces mortality in endotoxemia. Proc Natl Acad Sci USA 104(41):16239-16244.
50. Fliedner S M, et al. (2015) Potential therapeutic target for malignant paragangliomas: ATP synthase on the surface of paraganglioma cells. American journal of cancer research 5(4):1558-1570.
51. Kawai Y, Kaidoh M, Yokoyama Y, & Ohhashi T (2013) Cell surface F1/FO ATP synthase contributes to interstitial flow-mediated development of the acidic microenvironment in tumor tissues. American journal of physiology. Cell physiology 305(11):C1139-1150.
52. Li Z, et al. (2013) Cell-surface GRP78 facilitates colorectal cancer cell migration and invasion. The international journal of biochemistry & cell biology 45(5):987-994.
53. Yao X, et al. (2015) Cell Surface GRP78 Accelerated Breast Cancer Cell Proliferation and Migration by Activating STAT3. PLoS One 10(5):e0125634.
54. Starck S R, et al. (2016) Translation from the 5′ untranslated region shapes the integrated stress response. Science (New York, N Y) 351(6272):aad3867.
55. Kuo L J, Hung C S, Chen W Y, Chang Y J, & Wei P L (2013) Glucose-regulated protein 78 silencing down-regulates vascular endothelial growth factor/vascular endothelial growth factor receptor 2 pathway to suppress human colon cancer tumor growth. The Journal of surgical research 185(1):264-272.
56. Liu R, et al. (2013) Monoclonal antibody against cell surface GRP78 as a novel agent in suppressing PI3K/AKT signaling, tumor growth, and metastasis. Clin Cancer Res 19(24): 6802-6811.
57. Carmi Y, et al. (2015) Allogeneic IgG combined with dendritic cell stimuli induce antitumour T-cell immunity. Nature 521(7550): 99-104.

1. A method for inducing endogenous antitumor autoantibodies (EAA) in a subject having a neoplasia comprising administering to the subject an effective amount of the nordihydroguaiaretic acid (NDGA) derivative P₄N, having the following formula: 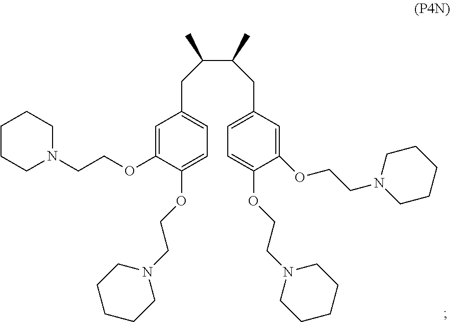 [SMILES: C[C@H](Cc1ccc(OCCN2CCCCC2)c(OCCN2CCCCC2)c1)[C@@H](C)Cc1ccc(OCCN2CCCCC2)c(OCCN2CCCCC2)c1]
or salts, solvates and stereoisomers thereof.

2. A method for inducing B cell proliferation in a subject having a neoplasia comprising administering to the subject an effective amount of the nordihydroguaiaretic acid (NDGA) derivative P₄N, having the following formula: 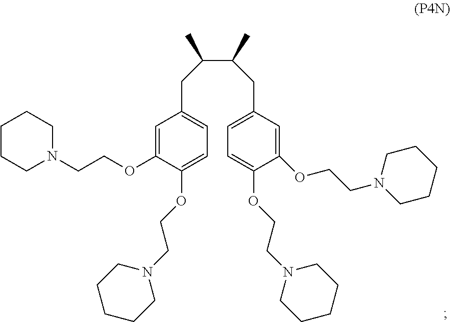 [SMILES: C[C@H](Cc1ccc(OCCN2CCCCC2)c(OCCN2CCCCC2)c1)[C@@H](C)Cc1ccc(OCCN2CCCCC2)c(OCCN2CCCCC2)c1]
or salts, solvates and stereoisomers thereof.

3. A method for inducing BAFF stimulated B cell proliferation in a subject having a neoplasia comprising administering to the subject an effective amount of the nordihydroguaiaretic acid (NDGA) derivative P₄N, having the following formula: 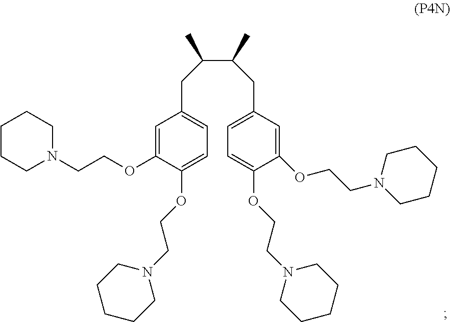 [SMILES: C[C@H](Cc1ccc(OCCN2CCCCC2)c(OCCN2CCCCC2)c1)[C@@H](C)Cc1ccc(OCCN2CCCCC2)c(OCCN2CCCCC2)c1]
or salts, solvates and stereoisomers thereof.

4. A method for suppressing or inhibition growth of a neoplasia in a subject having a neoplasia comprising administering to the subject an effective amount of a pharmaceutical composition comprising the nordihydroguaiaretic acid (NDGA) derivative P₄N, having the following formula: 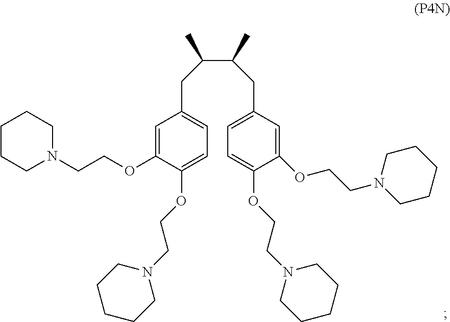 [SMILES: C[C@H](Cc1ccc(OCCN2CCCCC2)c(OCCN2CCCCC2)c1)[C@@H](C)Cc1ccc(OCCN2CCCCC2)c(OCCN2CCCCC2)c1]
or salts, solvates and stereoisomers thereof, and a pharmaceutically acceptable carrier.

5. The method of claim 1, wherein an effective amount of P₄N is a dose of about 0.1 mg/kg to about 10 mg/kg.

6. The method of claim 5, wherein the effective amount of P₄N is a dose of about 2.5 mg/kg to about 5 mg/kg.

7. The method of claim 1, wherein the neoplasia is cancer and micro and macro metastases.

8. The method of claim 7, wherein the cancer is colorectal cancer.

9. The method of claim 1, wherein the method further comprises administration to the subject at least one additional biologically active agent.

10. The method of claim 9, wherein the at least one additional biologically active agent is an anticancer agent.

11. The method of claim 2, wherein an effective amount of P₄N is a dose of about 0.1 mg/kg to about 10 mg/kg.

12. The method of claim 11, wherein the effective amount of P₄N is a dose of about 2.5 mg/kg to about 5 mg/kg.

13. The method of claim 2, wherein the neoplasia is cancer and micro and macro metastases.

14. The method of claim 13, wherein the cancer is colorectal cancer.

15. The method of claim 2, wherein the method further comprises administration to the subject at least one additional biologically active agent.

16. The method of claim 15, wherein the at least one additional biologically active agent is an anticancer agent.

17. The method of claim 3, wherein an effective amount of P₄N is a dose of about 0.1 mg/kg to about 10 mg/kg.

18. The method of claim 17, wherein the effective amount of P₄N is a dose of about 2.5 mg/kg to about 5 mg/kg.

19. The method of claim 3, wherein the neoplasia is cancer and micro and macro metastases.

20. The method of claim 19, wherein the cancer is colorectal cancer.

21. The method of claim 3, wherein the method further comprises administration to the subject at least one additional biologically active agent.

22. The method of claim 21, wherein the at least one additional biologically active agent is an anticancer agent.

23. The method of claim 4, wherein an effective amount of P₄N is a dose of about 0.1 mg/kg to about 10 mg/kg.

24. The method of claim 23, wherein the effective amount of P₄N is a dose of about 2.5 mg/kg to about 5 mg/kg.

25. The method of claim 4, wherein the neoplasia is cancer and micro and macro metastases.

26. The method of claim 25, wherein the cancer is colorectal cancer.

27. The method of claim 4, wherein the method further comprises administration to the subject at least one additional biologically active agent.

28. The method of claim 27, wherein the at least one additional biologically active agent is an anticancer agent.
